(19) United States
(12) Patent Application Publication
(10) Pub. No.: US 2006/0160773 A1
(43) Pub. Date: Jul. 20, 2006

(54) Combretastatin derivatives with cytotoxic action

(76) Inventors: Giuseppe Giannini, Pomezia (IT); Claudio Pisano, Aprilia (IT); Domenico Alloatti, Roma (IT); Romeo Romagnoli, Ferrara (IT); Daniele Simoni, Ferrara (IT)

Correspondence Address: NIXON & VANDERHYE, PC, 901 NORTH GLEBE ROAD, 11TH FLOOR, ARLINGTON, VA, 22203, US

(21) Appl. No.: 10/563,465

(22) PCT Filed: Jul. 6, 2004
(86) PCT No.: PCT/IT04/00373

§ 371 Date: Jan. 5, 2006

(30) Foreign Application Priority Data
Jul. 18, 2003 (IT) RM2003A000355

(51) Int. Cl. C07D 261/02; A61K 31/42; A61K 31/40; A61K 31/404; A61K 31/381

(52) U.S. Cl. 514/92; 514/378; 514/114; 514/408; 514/415; 514/438; 514/471; 548/112; 548/240; 548/413; 548/516; 548/577

(57) ABSTRACT

The invention described herein relates to new combretastatin derivatives obtained by total synthesis and having the following general formula: 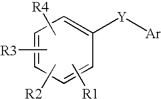 in which the groups are as defined in the description here below. Said compounds, though chemically related to the structure of cis/trans-combretastatin, do not always bind tubulin, but nevertheless exhibit cytotoxic activity of interest in the oncological field as anticancer and/or antiangiogenic agents.

The invention described herein relates to new combretastatin derivatives obtained by total synthesis, to processes for their preparation, to their use as medicaments and to compositions containing them.

The development strategy for each product has been selected from the group consisting of: (i) substitution of the olefinic bond with a heterocycle of the isoxazole or 4,5-dihydro-3-R-isoxazole type, or ii) substitution of one or both H's present on the olefinic bond with a fluorine and/or iii) substitution of an aromatic residue with an aromatic heterocyclic residue of the benzofuran, benzothiophene, indole and indazole, furan or thiophene type, or with naphthyl groups, with optionally functionalised substituent groups, and/or iv) substitution of one or more methoxy residues on the trimethoxyphenyl with other substituents. Said compounds, though chemically related to the structure of cis/trans-combretastatin, do not always bind tubulin, but nevertheless exhibit a cytotoxic activity of interest in the oncological field as anticancer or antiangiogenic agents.

Antitubulin activity is not regarded as an essential requisite for anticancer activity; in actual fact, the anticancer activity of combretastatin is the result of a series of pharmacodynamic- and pharmacokinetic-type components.

### BACKGROUND TO THE INVENTION

Angiogenesis in the adult is normally quiescent, yet it constitutes a normal function, for example in the healing of wounds or in the reconstruction of the endometrium during the female reproductive cycle. The angiogenic response is physiologically stimulated when the vascular functions are reduced and tissue perfusion inadequate.

More generally, it can be claimed that angiogenesis, in physiological conditions, constitutes a form of positive feedback in response to inadequate perfusion, or to a reduced supply of oxygen and nutrients, as, for instance, in the case of occlusion of an artery, in situations of growth of tissue mass (e.g. the neovascularisation that accompanies the formation of muscle tissue); and in the case of an increased work load associated with an increased oxygen and nutrient requirement. In the course of local ischaemia, due to partial or complete occlusion of an artery, the development of collateral vessels is necessary in order to maintain perfusion.

The growth of a primary tumour is known to be favoured by good vascularisation of the tumour tissue. An adequate supply of oxygen and nutrients favours the rapid growth of the tumour itself. It has been demonstrated that the extent of neoangiogenesis is a highly adverse factor in the prognosis of neoplasms (van Hinsbergh, V. W., Collen, A., Koolwijk, P.: Ann. On-col., 10 Suppl., 4:60-3, 1999; Buolamwini, J. K: Curr., Opin., Chem., Biol., 3(4):500-9, 1999).

Research directed towards the discovery of new-generation chemotherapeutic agents has identified tubulin as a possible cell target. Substances capable of altering microtubule aggregation are also capable of inhibiting cell proliferation.

The microtubules play a very important role in the regulation of the cell architecture, in cell division and in cell metabolism. The systems of the microtubules of eukaryotic cells include the dynamic organisation of the aggregation and disaggregation of the matrix in which tubulin heterodimers polymerise to form microtubules both in cancer cells and in normal cells. Cytotoxic agents capable of altering the polymerisation or depolymerisation of the microtubules prove to be effective chemotherapeutic agents.

Combretastatin A-4 (CA-4), isolated from a variety of African willow, Combretuin caffrum (Combretaceae) (Pettit, G. R. et al.: Experientia, 1989, 45, 209), exhibits promising anticancer potential with an antitubulin mechanism, strongly binding tubulin in a site very similar to that to which colchicine binds (Lin, C. N. et al.; Biochemistry, 1989, 28, 6984). Said binding to tubulin prevents its polymerisation to microtubules with an antimitotic effect. CA-4 inhibits cell growth even at very low concentrations, of the order of nanomoles.

The phosphate salt of CA-4—“CA-4P” (Pettit, G. R. et al.; Anti-cancer Drug Des. 1995, 10, 299),—is hydrosoluble and is currently inserted in phase II clinical trials.

The ability of combretastatin to selectively impair tumour neovascularisation makes this compound distinctly interesting and prompts the search for new and more potent compounds.

Recently, many studies have demonstrated that a substantial number of compounds with antiangiogenic activity, such as CA-4P, are capable of inhibiting the neovascularisation of the retina in well characterised murine models of forms of retinopathy. These studies suggest that both CA-4P and the new derivatives could be usefully employed as antiangiogenic agents in the fields of both oncology and ophthalmology (Griggs J. et al.: Am. J. Pathol. 2002, 160(3), 1097-103).

Nevertheless, the very substantial cytotoxic potency of combretastatin cannot be put down only to its antitubulin effect. There are compounds of analogous structure which, though exhibiting substantial cytotoxicity, do not exert an equally high degree of antitubulin activity.

In addition to the pharmacokinetic aspects, there are many pharmacodynamic aspects which are still the subject of thorough investigation, and, as things stand at present, there are not enough literature data available to furnish a definitive response (Le Wanzg et al.: J. Med. Chem, 2002, 45, 1697-1711).

From the chemical standpoint it is known that the distance between the two aromatic rings of combretastatin, colchicine or their derivatives constitutes an immutable requirement of this class of compounds for their antitubulin properties (McGown, A. T. et al.; a) Bioorg. Med. Chem. Lett., 1988, 8(9), 1051-6; b) Bioorg. Med. Chem. Lett. 2001, 11(1), 51-4).

Substitution of the double bond with an indolyloxazoline residue (Qun Li, Q. et al.: Bioorg. Med. Chem. Lett., 2002, 12(3), 465-9) has led to a combretastatin derivative, A-289099, (where an aromatic ring is also substituted with an N-Me-indole residue), with anticancer activity comparable to that of the comparator reference product.

Stilbene and dihydrostilbene derivatives that inhibit tubulin polymerization are described in Cushing et al. works (J. Med. Chem., 1991, 34, 2579-2588; 1992, 35, 2293-2306, U.S. Pat. No. 5,430,062), Woods et al. (British Journal of Cancer, 1995, 71, 705-711), U.S. Pat. No. 5,512,678, U.S. Pat. No. 5,525,632 and Ohsumi et al. (J. Med. Chem., 1998, 41, 3022-3032), Hatanaka et al. (Bioorganic & Medicinal Chemistry Letters, 1998, 8, 3371-3374), Maya et al. (Bioorganic & Medicinal Chemistry Letters, 2000, 10, 2549-2551), Li et al. (Bioorganic & Medicincal Chemistry Letters, 2002, 12, 465-469), Hori et al. (British Journal of Cancer, 2002, 86, 1604-1614), WO 02/50007, Pettit et al. (J. Med. Chem., 2003, 46(4), 525-531), Wang et al. (J. Med. Chem., 2002, 45, 1697-1711), Kim et al. (Chem. Pharm. Bull., 2003, 51(5), 516-521),

It is equally well known in the cancer field that a fundamental stage in the biology of tumour cells consists in their acquiring the ability to cause metastases.

Tumour cells that metastasise have the ability to lose adhesion to the surrounding structures, invade blood and lymph vessels and colonise other tissues at a distance where they then continue to reproduce.

Metastatic spread is also a critical event in the clinical history of the disease, being the main cause of death from cancer. It is closely associated with, and favoured by the presence of vascular tissue in the tumour site or in the adjacent areas.

In fact, cancer cell migration through the surrounding structures allows the cells to reach the blood vessels in the tumour, whether preexisting or formed by neoangiogenesis, and from where they then proceed to the bloodstream (Ray, J. M., Stetler-Stevenson, W. G.: Eur. Respir. J., 1994, 7(11):2062-72; Stetler-Stevenson, W. G., Liotta, L. A., Kleiner D. E. Jr.: FASEB J., 1993, 7(15):1434-41).

The presence of communication paths between lymphatic and blood vessels allows cancer cells to move in both vascular systems.

Recent studies have revealed a direct relationship between angiogenesis and arthritic disease (Koch, A. E.: Arthritis and Rheumatism, 1998, 41:951-962). In particular, it has been demonstrated that neovascularisation of the articular cartilages plays a crucial role in the formation of the pannus and in the progression of arthritis. A normal cartilage has no blood vessels, whereas the sinovial fluid of arthritic patients contains an angiogenesis-stimulating factor produced by the endothelial cells (endothelial-cell-stimulating angiogenesis factor=ESAF).

The presence of this factor is associated with the vascularisation and degradation of the cartilage.

Other diseases are also related to abnormal angiogenesis.

It has been found that neovascularisation of the affected tissues is a causative factor favouring diabetic retinopathy (Histol. Histopathol., 1999; 14(4):1287-94), psoriasis (Br. J. Dermatol., 1999 141(6):1054-60), chronic inflammation and atherosclerosis (Planta Med., 1998; 64(8):686-95).

Control of neovascularisation is therefore one of the fundamental elements for the control and treatment of such diseases.

Despite the progress made over the past few years in the field of new drugs endowed with antiangiogenic activity, this field of research is regarded by many experts in the field of medicament as still being one of the most promising for the discovery of new drugs for the treatment of diseases characterised by abnormal angiogenesis, particularly tumours.

In fact, for these diseases there is an even more strongly perceived need for new compounds presenting fewer side effects and which are capable of blocking or interfering with the abnormal mechanisms underlying the above-mentioned diseases and which therefore allow such diseases to be treated.

It has now surprisingly been found that by modifying both the double olefinic bond and the aromatic rings of combretastain, the result is the general formula (I) compounds described here below, with antitubulin and/or cytotoxic properties, which are useful agents for the treatment of diseases caused by abnormal angiogenesis and of tumours.

In a thoroughly unexpected manner, the derivatives according to the present invention show that the cytotoxic activity can still be very substantial even in the presence of low or non-existent antitubulin activity.

### SUMMARY OF THE INVENTION

One object of the present invention are formula (I) compounds 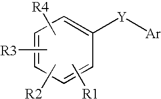
in which
the various R₁, R₂, R₃ and R₄, which can be the same or different, are H, OH, OPO₃H₂ or OCH₂OPO₃H₂ and their disodium salt, OMe, OCH₂O, NO₂, F, Cl, Br;
—R₁—R₂— can also be together: —CR₈═CR₉—X—
Y is a group selected from 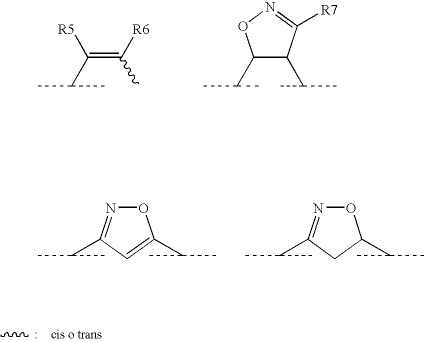
R₅ and R₆, which can be the same or different, are H or halogen;
R₇ is H, OMe, SO₂Ph;
Ar is a group selected from: 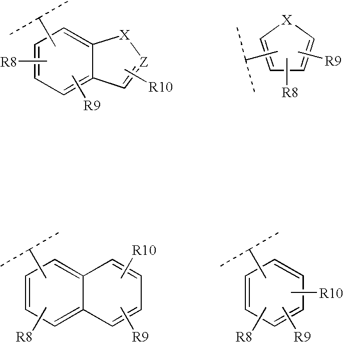
R₈, R₉ and R₁₀, which can be the same or different, are H, OH, OPO3H2 or OCH₂OPO₃H₂ and their disodium salt, OR₁₁, OCH₂O, NH₂, NHR₁₁, NO₂, alkyl (C₁-C₄), C₆ Hr, C₅H₄N or halogen;
R₁₁ is C₁-C₄ alkyl or acyl, amino acid residue;
X is O, S, N, NR₁₂;
R₁₂ is H, CH₃, CH₂Ph;
Z is CH, N;
with the proviso that the formula (I) compound is not combretastatin A-1, combretastatin A-2, combretastatin A-4, and their disodium phosphates derivatives and with the exclusion of the following compounds:
2-phenyl-6-trans-styryl-benzo[b]furan;
2,3-diphenyl-6-trans-styryl-benzo[b]furan;
2-phenyl-6-(4-methoxy)-trans-styryl-benzo[b]furan;
2-phenyl-6-(3,4-dimethoxy)-trans-styryl-benzo[b]furan;
2-phenyl-6-(3,4,5-trimethoxy)-trans-styryl-benzo[b]furan;
2-phenyl-6-(3,4-methylenedioxy)-trans-styryl-benzo[b]furan;
2,3-diphenyl-6-(4-methoxy)-trans-styryl-benzo[b]furan;
2-phenyl-5-trans-styryl-benzo[b]thiophene;
2-phenyl-5-(4-methoxy)-trans-styryl-benzo[b]thiophene;
2-phenyl-5-(3,4-methylenedioxy)-trans-styryl-benzo[b]thiophene;
2-phenyl-6-trans-styryl-benzo[b]thiophene; 2-phenyl-6-(4-methoxy)-trans-styryl-benzo[b]thiophene;
2-phenyl-6-(4-chloro)-trans-styryl-benzo[b]thiophene; Piceatannol; 1-(3-furanyl)-2-(3,4,5-trimethoxyphenyl)ethene;
1-(3-thiophenyl)-2-(3,4,5-trimethoxyphenyl)ethene;
1-(2-furanyl)-2-(3,4,5-trimethoxyphenyl)ethene; and with the proviso that

when R₁ is hydrogen and R₂-R₄ are 3,4,5-trimethoxy, Y is a double bond, R₅ and R₆ are H, Ar is phenyl, R₈ and R₅ are hydrogen, R₁₀ is not methoxy;

when R₁ is hydrogen and R₂-R₄ are 3,4,5-trimethoxy, Y is a double bond, R₅ and R₆ are H, Ar is phenyl, R₈ is hydrogen, R₉ is 2-chloro, R₁₀ is not 4-methoxy;

when R₁ is hydrogen and R₂-R₄ are trimethoxy, Y is a double bond, R₅ and R₆ are H, Ar is phenyl, at least one of R₈-R₁₀ is not hydrogen;

when R₁ is hydrogen and R₂-R₄ are 3,4,5-trimethoxy, Y is a double bond, R₅ and R₆ are H, Ar is phenyl, R₈ and R₉ are hydrogen, R₁₀ is none of 4-chloro, 4-bromo, 4-nitro, 4-hydroxy, 4-acetyl, 4-ethoxy, 4-C₁-C₄ alkyl;

when R₁ is hydrogen and R₂-R₄ are 3,4,5-trimethoxy, Y is a double bond, R₅ and R₆ are H, Ar is phenyl, R₈ is hydrogen, R₉ is 4-nitro or 4-amino, R₁₀ is none of 3-chloro, 3-methoxy, 3-methyl;

when R₁ is hydrogen and R₂-R₄ are 3,4,5-trimethoxy, Y is a cis double bond, R₅ and R₆ are H, Ar is phenyl, R₈ is hydrogen, R₉ is 3-nitro or 3-amino, R₁₀ is none of 3-chloro, 3-methoxy, 3-methyl;

when R₁ is hydrogen and R₂-R₄ are 2,3,4-trimethoxy, Y is a double bond, R₅ and R₆ are H, Ar is phenyl, R₈ and R₉ are hydrogen, R₁₀ is not 4-methoxy;

when R₁ is hydrogen and R₂-R₄ are 3,4,5-trimethoxy, Y is a double bond, R₅ and R₆ are H, Ar is phenyl, at least one of R₈ is hydrogen, R₉ is 3-methoxy, R₁₀ is not 5-methoxy;

when R₁ is hydrogen and R₂-R₄ are 3,4,5-trimethoxy, Y is a double bond, R₅ and R₆ are H, Ar is phenyl, R₈-R₁₀ are not methoxy;

when R₁ and R₂ are hydrogen and R₃-R₄ are 3,4-dimethoxy, Y is a double bond, R₅ and R₆ are H, Ar is phenyl, R₈ and R₉ are hydrogen, R₁₀ is not 4-methoxy;

when R₁ and R₂ are hydrogen and R₃-R₄ are 3,4-dimethoxy, Y is a double bond, R₅ and R₆ are H, Ar is phenyl, R₈ is hydrogen, R₉-R₁₀ are not 3,5-dimethoxy;

when R₁ and R₂ are hydrogen and R₃-R₄ are 3,4-dimethoxy, Y is a double bond, R₅ and R₆ are H, Ar is phenyl, at least one of R₈-R₁₀ is not hydrogen;

when R₁ and R₂ are hydrogen and R₃-R₄ are 3,5-methoxy, Y is a double bond, R₅ and R₆ are H, Ar is phenyl, R₈ and R₅ are hydrogen, R₁₀ is not 4-methoxy;

when R₁ and R₂ are hydrogen and R₃-R₄ are 3,5-methoxy, Y is a double bond, R₅ and R₆ are H, Ar is phenyl, R₈ and R₅ are hydrogen, R₁₀ is not 4-acetyl;

when R₁ is hydrogen and R₂-R₄ are 3,4,5-trimethoxy, Y is a double bond, R₅ and R₆ are H, Ar is not pyridyl;

when R₁ is hydrogen and R₂-R₄ are 3,4,5-trimethoxy, Y is cis double bond, R₅ and R₆ are H, Ar is phenyl, R₈ is hydrogen, R₅ is 3-amino, R₁₀ is 4-NHR₁₁, R₁₁ is not the residue of serine;

when R₁ is hydrogen and R₂-R₄ are 3,4,5-trimethoxy, Y is cis double bond, R₅ and R₆ are H, Ar is phenyl, R₈ is hydrogen, R₅ is 3-amino, R₁₀ is not 4-methoxy;

when R₁ is hydrogen and R₂-R₄ are 3,4,5-trimethoxy, Y is cis double bond, R₅ and R₆ are H, Ar is phenyl, R₈ is hydrogen, R₅ is 3-amino, R₁₀ is not a 4-alkyloxy group having from 1 to 3 carbon atoms, or a 4-alkyl group having from 1 to 4 carbon atoms, or a halogen atom

when R₁ is hydrogen and R₂-R₃ are 3,4-methylenedioxy, R₄ is 5-methoxy, Y is cis double bond, R₅ and R₆ are H, Ar is phenyl, R₈ is hydrogen, R₅ is 3-amino, R₁₀ is not 4-methoxy;

when R₁ is hydrogen and R₂-R₄ are 2,3,4-trimethoxy, Y is cis double bond, R₅ and R₆ are H, Ar is phenyl, R₈ is hydrogen, R₉ is 3-amino, R₁₀ is not 4-methoxy;

when R₁ is hydrogen and R₂-R₄ are 3,4,5-trimethoxy, Y is cis double bond, R₅ and R₆ are H, Ar is phenyl, R₈ is hydrogen, R₉ is NHR₁₁, R₁₁ is the residue of serine, R₁₀ is not 4-methoxy;

when R₁ is hydrogen and R₂-R₃ are 3,4-methylenedioxy, R₄ is 4-methoxy, Y is cis double bond, R₅ and R₆ are H, Ar is phenyl, R₈ is hydrogen, R₉ is NHR₁₁, R₁ is the residue of the aminoacid cysteine, glycine, phenylalanine, serine, triptophan, tyrosine, valine, R₁₀ is not 4-methoxy;

when R₁ is hydrogen and R₂-R₃ are 3,4-methylenedioxy, R₄ is 4-methoxy, Y is a double bond, R₅ and R₆ are H, Ar is phenyl, R₈ is hydrogen, R₉ is NO₂ or NH₂, R₁₀ is not 4-methoxy;

when R₁ is hydrogen and R₂-R₄ are 3,4,5-trimethoxy, Y is cis double bond, R₅ and R₆ are H, Ar is phenyl, at least one of R₈-R₁₀ is not hydrogen;

when R₁ is hydrogen and R₂-R₄ are 3,4,5-trimethoxy, Y is a double bond, R₆ and R₆ are H, Ar is phenyl, R₈ is hydrogen, R₉ is 4-methoxy, R₁₀ is not 3-fluoro;

when R₁ is hydrogen and R₂-R₄ are 3,4,5-trimethoxy, Y is a double bond, R₅ and R₆ are H, Ar is phenyl, R₈ is hydrogen, R₉ is 4-methyl, R₁₀ is not 3-fluoro or 3-hydroxy;

when R₁ is hydrogen and R₂-R₄ are 3,4,5-trimethoxy, Y is cis double bond, R₅ and R₆; are H, Ar is phenyl, R₅ is hydrogen, R₉ is 4-methoxy, R₁₀ is not 3-methoxy;

when R₁ is hydrogen and R₂-R₄ are 3,4,5-trimethoxy, Y is cis double bond, R₅ and R₆ are H, Ar is phenyl, R₈ is 3-fluoro, R₅ is 4-methoxy, R₁₀ is not 2- or 5-fluoro;

when R₁ is hydrogen and R₂-R₄ are 3,4,5-trimethoxy, Y is cis double bond, R₅ and R₆ are H, Ar is phenyl, R₈ is hydrogen, R₅ is 4-methoxy, R₁₀ is not 3-hydroxy or 3-amino;

when R₁ is hydrogen and R₂-R₄ are 3,4,5-trimethoxy, Y is cis double bond, R₅ and R₆ are H, Ar is phenyl, R₈ is hydrogen, R₅ is 4-methoxy, R₁₀ is not 3-fluoro or 3-bromo;

when R₁ is hydrogen and R₂-R₄ are 3,4,5-trimethoxy, Y is cis double bond, R₅ and R₆ are H, Ar is phenyl, R₈ and R₉ are hydrogen, R₁₀ is not 4-hydroxy;

when R₁ is hydrogen and R₂-R₄ are 3,4,5-trimethoxy, Y is cis double bond, R₅ and R₆ are H, Ar is phenyl, R₈ is hydrogen, R₉ is 3-methyl, R₁₀ is not 4-methyl;

when R₁ is hydrogen and R₂-R₄ are 3,4,5-trimethoxy, Y is cis double bond, R₅ and R₆ are H, Ar is phenyl, R₈ is hydrogen, R₉ is 4-methoxy, R₁₀ is not 3-hydroxy;

when R₁-R₂ are hydrogen and R₃-R₄ are 3,5-dihydroxy, Y is trans double bond, R₅ and R₆ are H, Ar is phenyl, R₈ is hydrogen, R₉ is 3-hydroxy, R₁₀ is not 5-hydroxy;

when R₁-R₃ are hydrogen, Y is a double bond, R₆ and R₆ are H, Ar is phenyl, R₈ is hydrogen, R₉ and R₁₀ are 3,4-dimethyl, and R₄ is not 4-methoxy;

when R₁-R₂ are hydrogen, Y is a double bond, R₅ and R₆ are H, Ar is phenyl, R₈ is hydrogen, R₉ and R₁₀ are 3,4-dimethyl, R₄ is 4-methoxy, R₃ is not 3-fluoro or 3-bromo or 3-nitro or 3-hydroxy;

when R₁-R₂ are hydrogen, Y is a double bond, R₅ and R₆ are H, Ar is phenyl, R₈-R₁₀ are 3,4,5-triethoxy, R₄ is 4-methoxy, R₃ is not 3-fluoro or 3-chloro or 3-bromo or 3-hydroxy;

when R₁-R₂ are hydrogen, R₄ is 4-methoxy, Y is a double bond, R₅ and R₆ are H, Ar is phenyl, R₈-R₉ are 4,5-dimethoxy, R₁₀ is 3-hydroxy, R₃ is not 3-fluoro or 3-hydroxy;

when R₁-R₂ are hydrogen, R₄ is 4-methoxy, Y is a double bond, R₅ and R₆ are H, Ar is phenyl, R₈-R₉ are 4,5-dimethoxy, R₁₀ is 3-methoxy, R₃ is not 3-fluoro;

when R₁ is hydrogen, R₂-R₄ are 3,4,5-trimethoxy, Y is a double bond, R₅ and R₆ are H, Ar is 2-naphthyl, at least one of R₈-R₁₀ is not hydrogen;

when R₁ and R₂ are hydrogen, R₃ is 3-hydroxy, R₄ is 4-methoxy, Y is a double bond, R₆ and R₆ are H, Ar is 2-naphthyl, at least one of R₈-R₁₀ is not hydrogen;

when R₁ is hydrogen, R₂-R₄ are 3,4,5-trimethoxy,
Y is 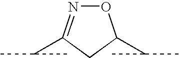
Ar is indolyl, wherein at least one of R₈-R₁₀ is different from hydrogen; their enantiomers, diastereoisomers, the respective mixtures and their pharmaceutically acceptable salts.

The invention relates to the use in the medical field as medicaments of new formula (I) compounds.

A further object of the present invention are pharmaceutical compositions containing as their active ingredient a formula (I) compound and at least one pharmaceutically acceptable excipient or diluent.

A further object of the present invention is the use of a formula (I) compound for the preparation of a medicament possessing cytotoxic-type anticancer activity.

A further object of the present invention is the use of a formula (I) compound for the preparation of a medicament with antiangiogenic-type anticancer activity.

A further object of the present invention is the use of a formula (I) compound for the preparation of a medicament useful for the prevention and reduction of cancer metastases.

A further object of the present invention is the use of formula (I) compounds for the preparation of a medicament with anticancer activity, in which the cancer is selected from the group consisting of: sarcoma, carcinoma, carcinoid, bone cancer, endocrine cancer, lymphoid leukaemia, myeloid leukaemia, monocytic leukaemia, megakaryocytic leukaemia, or Hodgkin's disease.

A further object of the present invention is the use of a formula (I) compound for the preparation of a medicament for the treatment of diseases related to abnormal angiogenesis in which the disease is selected from the group consisting of arthritic disease, tumours, metastatic spread, diabetic retinopathy, psoriasis, chronic inflammation, and atherosclerosis.

### DETAILED DESCRIPTION OF THE INVENTION

According to the present invention, pharmaceutically acceptable salts are all those salts that the expert in the field is capable of preparing, without the acid or base utilised giving rise to unwanted side effects, when said salts are used as medicaments.

Particularly preferred compounds are:
2-methoxy-5-[3-methoxy-5-(3,4,5-trimethoxy-phenyl)-4,5-dihydro-4-isoxazolyl]-phenol—ST1996;
2-methoxy-5-[3-methoxy-4-(3,4,5-trimethoxy-phenyl)-4,5-dihydro-5-isoxazolyl]-phenol—ST1998;
5-[3-benzenesulphonyl-4-(3,4,5-trimethoxy-phenyl)-4,5-dihydro-4-isoxazolyl]-2-methoxy-phenol—ST1995;
5-[3-benzenesulphonyl-5-(3,4,5-trimethoxy-phenyl)-4,5-dihdro-5-isoxazolyl]-2-methoxy-phenol—ST1997;
2-methoxy-5-[3-(3,4,5-trimethoxy-phenyl)-4,5-dihydro-5-isoxazolyl]-phenol—ST1999;
2-methoxy-5-[5-(3,4,5-trimethoxy-phenyl)-4,5-dihydro-3-isoxazolyl]-phenol—ST2001;
2-methoxy-5-[5-(3,4,5-trimethoxy-phenyl)-3-isoxazole]-phenol—ST2002;
cis-6-[2-(3,4,5-trimethoxy-phenyl)-vinyl]-benzo[b]thiophen-4-ol—ST2151;
trans-6-[2-(3,4,5-trimethoxy-phenyl)-vinyl]-benzo[b]thio-phen-4-ol—ST2152;
cis-4-methoxy-6-[2-(3,4,5-trimethoxy-phenyl)-vinyl]-benzo[b]thiophene—ST2049;
trans-4-methoxy-6-[2-(3,4,5-trimethoxy-phenyl)-vinyl]-benzo[b]thiophene—ST2050;
cis-6-[2-(3,4,5-trimethoxy-phenyl)-vinyl]-benzofuran-4-ol—ST2179;
trans-6-[2-(3,4,5-trimethoxy-phenyl)-vinyl]-benzofuran-4-ol—ST2180;
cis-4-methoxy-6-[2-(3,4,5-trimethoxy-phenyl)-vinyl]-benzofuran—ST2051;
trans-4-methoxy-6-[2-(3,4,5-trimethoxy-phenyl)-vinyl]-benzofuran—ST2052;
cis-5-[2-(3,4,5-trimethoxy-phenyl)-vinyl]-benzo[b]thiophen-7-ol—ST2487;
trans-5-[2-(3,4,5-trimethoxy-phenyl)-vinyl]-benzo[b]thiophen-7-ol—ST2488;
cis-5-[2-(3,4,5-trimethoxy-phenyl)-vinyl]-benzofuran-7-ol—ST2491;
trans-S-[2-(3,4,5-trimethoxy-phenyl)-vinyl]-benzofuran-7-ol—ST2492;
cis-1-methoxy-3-[2-(3,4,5-trimethoxy-phenyl)-vinyl]-naphthalene—ST2053;
methoxy-3-[2-(3,4,5-trimethoxy-phenyl)-vinyl]-naphthalene—ST2054;
cis-7-methoxy-1-methyl-5-[2-(3,4,5-trimethoxy-phenyl)-vinyl]-1H-indazole—ST2055;
trans-7-methoxy-1-methyl-5-[2-(3,4,5-trimethoxy-phenyl)-vinyl]-1H-indazole—ST2056;
2-nitro-5-[2-(3,4,5-trimethoxy-phenyl)-vinyl]-thiophene—ST2057;
2-nitro-5-[2-(3,4,5-trimethoxy-phenyl)-vinyl]-furan—ST2058;
cis-3-[2-(3,4,5-trimethoxy-phenyl)-vinyl]-naphthalen-1-ol—ST2181;
trans-3-[2-(3,4,5-trimethoxy-phenyl)-vinyl]-naphthalen-1-ol—ST2182;
disodium 6[(Z)-2-(3,4,5-trimethoxy-phenyl)ethenyl]-1-benzo-thiophen-4-ol 4-O-phosphate—ST2495;
disodium 6[(Z)-2-(3,4,5-trimethoxyphenyl)ethenyl]-1-benzo-furan-4-ol 4-O-phosphate,—ST2496;
6-[(Z)-2-(7-methoxy-1,3-benzodioxol-5-yl)vinyl]-1-benzothiophene-4-ol—ST2892;
6-[(E)-2-(7-methoxy-1,3-benzodioxol-5-yl)vinyl]-1-benzothiophene-4-ol—ST2891.
6[(Z)-2-(3-methoxy-4,5-metilendioxy-phenyl-1-yl)-vinyl]-1-benzofuran-4-ol—ST2933;
6[(E)-2-(3-methoxy-4,5-methylenedioxy-phenyl-1-yl)-vinyl]-1-benzofuran-4-ol—ST2934;
disodium 6[(Z)-2-(3,4,5-trimethoxy-phenyl)ethenyl]-1-benzo-thiophen-4-ol 4-O-methyloxyphosphate;
disodium 6[(Z)-2-(3,4,5-trimethoxyphenyl)ethenyl]-1-benzo-furan-4-ol 4-O-methyloxyphosphate;
6-[(Z)-2-(7-methoxy-1,3-benzodioxol-5-yl)vinyl]-1-benzothiophene-4-ol—ST2892;
cis-2-Methoxy-5-[2-(4-methoxy-benzofuran-6-yl)-vinyl]-phenol ST2897;
cis-2-Methoxy-5-[2-(7-methoxy-benzofuran-5-yl)-vinyl]-phenol, ST2898;
cis-2-Methoxy-5-[2-(4-methoxy-benzo[b]thiophen-6-yl)-vinyl]-phenol—ST2899;
cis-6-[2-(3,5-dimethoxy-phenyl)-vinyl]-benzo[b]thiophen-4-ol—ST2900;
cis-5-[2-(3,5-dimethoxy-phenyl)-vinyl]-benzofuran-7-ol—ST2901;
cis-6-[2-(3,5-dimethoxy-phenyl)-vinyl]-benzofuran-4-ol—ST2902.

The compounds described in this invention were prepared according to synthesis Schemes 1-15.

In particular, the formula (I) compounds in which Y is the isoxazoline ring and R₇ is a phenylsulphonic residue, such as, for example, the compounds called ST1995 and ST1997, were prepared according to Scheme 1 through the dipolar cycloaddition reaction [3+2]-of the nitryloxide generated by nitroderivative 2 on suitably protected combretastatin. Removal of the protective group, such as terbutyl-dimethylsilyl, leads to the desired compounds ST1995 and ST1997.

On the other hand, in those cases in which the R₇ group is methoxy, as, for example, in the compounds called ST1996 and ST1998, the compounds are obtained through the substitution of the phenylsulphonic group, as in the previous compounds ST1995 and ST1997, by means of reaction with sodium methoxylate.

The regioisomeric isoxazoline derivatives, such as, for example, ST1999 and ST2001, were prepared according to synthesis Schemes 2 and 3 through the dipolar cycloaddition reactions [3+2]-between nitryloxides generated by oximes 5 and 10 and the alkene components 6 and 9, respectively. Removal of the terbutyl-dimethylsilyl protective group leads to the desired products.

The regioisomeric isoxazole derivatives, such as, for example ST2000 and ST2002, were in turn prepared through the manganese-dioxide-mediated oxidation of the isoxazolines described above, suitably protected according to synthesis Schemes 2 and 3. Removal of the protective group, such as terbutyl-dimethylsilyl, leads to the desired products.

The formula (I) compounds in which Ar is a benzothiophene or benzofuran residue, such as, for example, compounds ST2151, ST2152, ST2049, ST2050, ST2179, ST2180, ST2051, ST2052, ST2487, ST2488, ST2491 and ST2492, were obtained according to the synthesis processes described in synthesis Schemes 4 and 5.

In particular, the Wittig reaction between aldehydes 17a-d and phosphonium salt 18, followed by removal of the ter-butyl-dimethylsilyl protective group, made it possible to obtain the desired derivatives (Scheme 4). In the same way, the Wittig reaction between aldehydes 26a,b and phosphonium salt 18, followed by removal of the appropriate protective group, such as terbutyl-dimethylsilyl, made it possible to obtain the desired derivatives, for example, ST2487, ST2488, ST2491 and ST2492 (Scheme 5).

A similar process was used to prepare the derivatives in which Ar is a naphthalene, indazole, nitrothiophene or nitrofuran residue, such as, for example, ST2053, ST2054, ST2055, ST2056, ST 2181, ST2057, ST2058, and ST2182 (Scheme 6) through the Wittig reaction between the appropriate aldehydes 29a-d and phosphonium salt 18.

Lastly, the formula (I) compounds in which R₈ or R₅ are a phosphate group, such as, for example, ST2495 and ST2496, were obtained according to the synthesis process described in Scheme 7 starting from the corresponding phenol derivatives, such as, for example, ST2151 and ST2179.

Other prodrug forms and/or more hydrosoluble derivatives were obtained according to the synthesis process described in Schemes 12-13 starting from the corresponding phenol or amine derivatives.

In the medical field the use is known of therapeutic protocols involving the administration of more than one anticancer drug simultaneously or in sequence, for example, as a function of the synchronisation of the cell cycles, with which experts in oncology are thoroughly familiar.

The need to administer more than one anticancer drug in therapeutic protocols is due to the fact that the drugs, by acting at different metabolic levels, favour, in some cases, complete remission of the cancer, and in other cases lengthen the life and/or improve the quality of life of the patient treated. The combination according to the present invention lends itself to use concomitantly with one or more known anticancer drugs for the treatment of tumours.

A further object of the present invention is therefore the use of formula (I) compounds, whether alone or in combination with other known antiblastic drugs, selected from the group consisting of: alkylating agents; topoisomerase inhibitors; antitubulin agents; intercalating agents; antimetabolites; naturally occurring products such as Vinca alkaloids, epipodophyllotoxins, antibiotics, enzymes, taxanes and anticancer vaccines.

The following examples further illustrate the invention.

Abbreviations used in the experimental part: TBDMSiCl (tertbutyldimethylchlorosilane); TBAF (tetra-n-butylammonium fluoride); NCS(N-chlorosuccinimide); Hex (Hexane); DAST (Diethylaminosulfur trifluoride); DIPEA (diisopropylethylamine); PyBroP (Bromo-tris-pyrrolidino-phosphonium-hexafluoro-phospate); TAEA (tris(2-aminoethyl)amine); BTMS (bromotrimethylsilane).

### EXAMPLE 1

Preparation of ST1995, ST1996, ST1997 and ST1998

These compounds are prepared according to synthesis Scheme 1 here below: 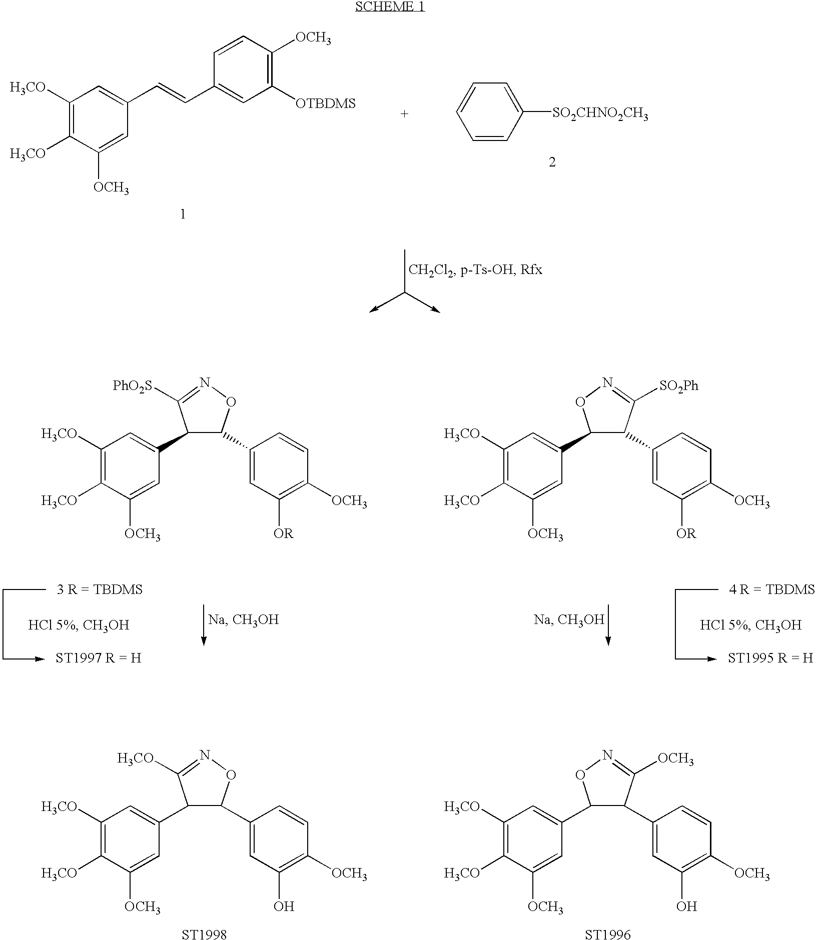
Preparation of Isoxazolines 3 and 4

To the flask containing nitronic ester 2 prepared according to the process described by Wade et al. (J. Org. Chem. 1981, 46, 765-770) is added alkene 1 (600 mg, 1.4 mmol) dissolved in CH₂Cl₂ (6 ml) and p-toluenesulphonic acid monohydrate (270 mg, 1.4 mmol). The reaction is refluxed for 30 minutes in an argon atmosphere. After bringing the solution back to room temperature CH₂Cl₂ (15 ml) is added, and washings are performed with 5% NaOH (10 ml), H₂O (10 ml) and brine (10 ml). The organic phase, anhydrified on Na₂SO₄, is evaporated at reduced pressure. Chromatographic purification of the crude product made it possible to obtain products 3 and 4 with an overall yield of 20%.

Preparation of ST1996 and ST1998

Metallic Na (130 mg, 0.6 mmol) is dissolved in MeOH (10 ml), the solution thus obtained is added to the appropriate phenyl-sulphonyl derivative 3, 4 (0.15 mmol) and the reaction is left at room temperature for 6 h.

After concentrating the ethanol and diluting with CH₂Cl₂ (15 ml), extractions are performed with H₂O (8 ml) and brine (8 ml). The organic solution, anhydrified on Na₂SO₄, is evaporated at reduced pressure.

The crude product obtained is purified by chromatography.

### 2-Methoxy-5-[3-methoxy-5-(3,4,5-trimethoxy-phenyl)-4,5-dihydro-4-isoxazol]-phenol—ST1996

Yield: 70%, m.p.=160-162° C.;

¹HNMR (CDCl₃) δ 3.84 (s, 9H), 3.91 (s, 6H), 4.19 (d, 1H, J=9.2 Hz), 5.38 (d, 1H, J=9.3 Hz), 5.69 (s, 1H), 6.54 (s, 2H), 6.69-6.74 (m,1H), 6.84-6.88 (m, 2H).

### 2-Methoxy-5-[3-methoxy-4-(3,45-trimethoxy-phenyl)-4,5-dihydro-5-isoxazolyl]-phenol—ST1998

Yield: 65%. Oil.

¹HNMR (CDCl₃) δ 3.86 (s, 9H), 3.91 (s, 6H), 4.14 (d, 1H, J=9.1 Hz), 5.40 (d, 1H, J=9.1 Hz), 5.68 (br, 1H), 6.43 (s, 2H), 6.84 (s, 2H), 6.95 (s, 1H).

Preparation of ST1995, ST1997

The appropriate silyl derivative (0.1 mmol) 3,4 is dissolved in MeOH (10 ml), and H₂O (½ ml) and HCl 5% (10 drops) are added to the solution. After being left overnight at room temperature, the methanol is evaporated, the crude product is extracted with CH₂Cl₂ (15 ml), and washed with H₂O (10 ml) and brine (10 ml). The organic solution, anhydrified and evaporated to dryness, produces a crude product that is purified by chromatography on silica gel.

### 5-[3-Benzenesulphonyl-4-(3,4,5-trimethoxy-phenyl)-4,5-dihydro-4-isoxazolyl]-2-methoxy-phenol—ST1995

Yield: 95%. Oil.

¹HNMR (CDCl₃) δ 3.67 (s, 6H), 3.82 (s, 3H), 3.91 (s, 3H), 4.58 (d, 1H, J=6.5 Hz), 5.56 (d, 1H, J=6.5 Hz), 5.62 (br, 1H), 6.15 (s, 2H), 6.79-6.84 (m, 3H), 7.37-7.43 (m, 2H), 7.55 (d, 1H, J=8.1 Hz), 7.61-7.65 (m,2H).

### 5-[3-Benzenesulphonyl-5-(3,4,5-trimethoxy-phenyl)-4,5-dihydro-5-isoxazolyl]-2-methoxy-phenol—ST1997

Yield: 85%. Oil.

¹HNMR (CDCl₃) δ 3.82 (s, 6H), 3.84 (s, 3H), 3.89 (s, 3H), 4.56 (d, 1H, J=6.6 Hz), 5.55 (d, 1H, J=6.5 Hz), 5.57 (br, 1H), 6.39 (s, 2H), 6.56-6.58 (m, 1H), 6.62 (d, 1H, J=2.1 Hz), 6.71 (d, 1H, J=8.1 Hz), 7.37-7.44 (m, 2H), 7.55-7.59 (m, 1H), 7.66-7.72 (m, 2H).

### EXAMPLE 2

Preparation of ST1999, ST2000, ST2001 and ST2002

These compounds are prepared according to synthesis Schemes 2 and 3 here below: 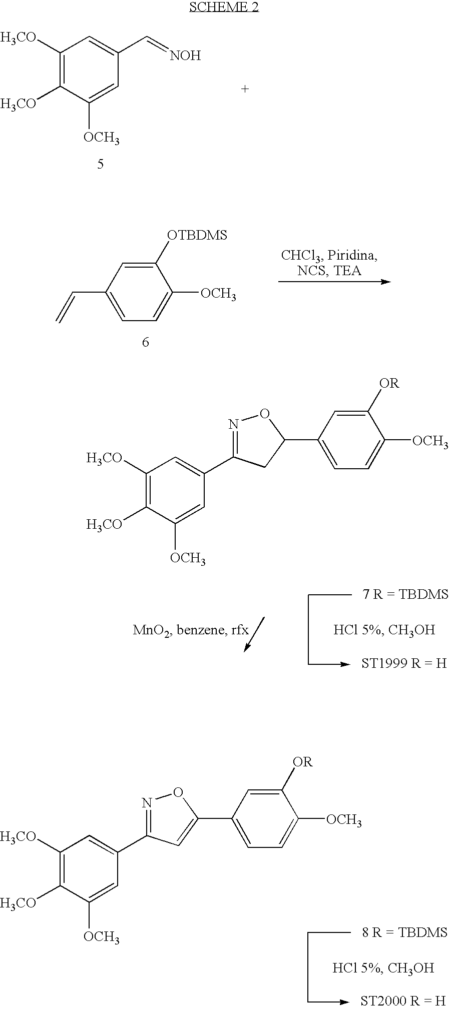 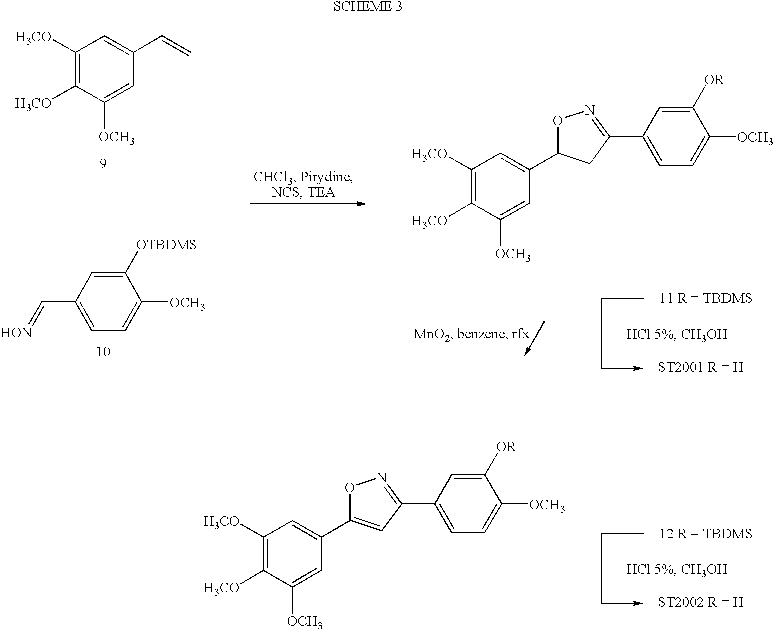
General Process for Preparation of 7 and 11.

To a flask containing anhydrous CHCl₃ (7 ml) are added NCS (1 mmol, 133 mg), pyridine (0.1 mmol, 7.9 mg, 8 μl) and the appropriate oxime 5, 10 (1 mmol). The reaction is stirred at 50° C. for 1 h. The corresponding alkene 6, 9 (1.1 mmol) is then added at room temperature and TEA (1.5 mmol, 152 mg, 0.2 ml) is added dropwise very slowly. The reaction mixture is left to stir for 2 h. CH₂Cl₂ (20 ml) is then added, and washings are performed with H₂O (15 ml), 2.5% HCl (10 ml), H₂O (10 ml) and brine (10 ml). The organic phase is anhydrified on Na₂SO₄ and concentrated at reduced pressure. The crude reaction product is purified by chromatography to give the desired isoxazoline. Yield of the cycloaddition: 70-75%.

General Process for the Preparation of Isoxazoles 8 and 12

Isoxazoline 7, 11 (50 mg, 0.1 mmol) is dissolved in benzene (15 ml), MnO₂ (450 mg, 5.17 mmol) is added to the solution, and the mixture is refluxed with Dean-Stark for 6 h under vigorous stirring.

The reaction mixture, brought back to room temperature, is filtered on celite and the filtrate is concentrated at reduced pressure.

The crude product thus obtained is purified by chromatography to give the isoxazole derivative. Oxidation yield: 80-85%.

The final compounds ST1999, ST2000, ST2001 and ST2002 are obtained from the corresponding precursors 7, 8, 11 and 12 through desilylation performed as described above for ST1997 and ST1995.

### 2-Methoxy-5-[3-(3,4,5-trimethoxy-phenyl)-4,5-dihydro-5-isoxazolyl phenol—ST1999

Yield: 85%, Oil,

1H-NMR (CDCl₃) δ: 3.30 (dd, 1H, J=8.2 Hz, 16.2 Hz), 3.74 (dd, 1H, J=10.9 Hz, 16.3 Hz), 3.89 (s, 12H), 5.65 (dd, 1H, J=8.2 Hz, 10.8 Hz), 5.63 (br, 1H), 6.85-6.95 (m, 5H).

### 2-Methoxy-5-[3-(3,4,5-trimethoxy-phenyl)-5-isoxazolyl]-phenol—ST

Yield: 95%, m.p.: 183-185° C.,

¹H-NMR (CDCl₃) δ: 3.91 (s, 3H), 3.95 (s, 3H), 3.96 (s, 9H), 5.80 (br, 1H), 5.82 (s, 1H), 6.94 (d, 1H, J=8.9 Hz), 7.08 (s, 2H), 7.37-7.41 (m, 2H).

### 2-Methoxy-5-[5-(3,4,5-trimethoxy-phenyl)-4,5-dihydro-3-isoxazolyl]phenol—ST2001

Yield: 90%, m.p.: 128-130° C.,

¹H-NMR (CDCl₃) δ: 3.30 (dd, 1H, J=8.4 Hz, 16.2 Hz), 3.75 (dd, 1H, J=10.4 Hz, 16.4 Hz), 3.86 (s, 3H), 3.90 (s, 6H), 3.96 (s, 3H), 5.65 (dd, 1H, J=8.2 Hz, 10.2 Hz), 5.68 (br, 1H), 6.62 (s, 2H), 6.90 (d, 1H, J=8.1 Hz), 7.22 (dd, 1H, J=2.1 Hz, 8.2 Hz), 7.28 (d, 1H, J=2.1 Hz).

### 2-Methoxy-5-[5-(3,4,5-trimethoxy-phenyl)-3-isoxazole]-phenol—ST

Yield: 80%, m.p.: 205-206° C.,

¹H-NMR (CDCl₃) δ: 3.90 (s, 3H), 3.95 (s, 9H), 5.80 (br, 1H), 6.70 (s, 1H), 6.93 (d, 1H, J=8.3 Hz), 7.04 (s, 2H), 7.36 (d, 1H, J=2 Hz), 7.43 (dd, 1H, J=1.9 Hz, 8.2 Hz).

### EXAMPLE 3

Preparation of ST2151, ST2152, ST2179, ST2180, ST2049, ST2050, ST2051, ST2052, ST2487, ST2488, ST2491, ST2492 [and ST2900, ST2901, ST29021

These compounds are prepared according to synthesis Schemes 4 and 5 here below: 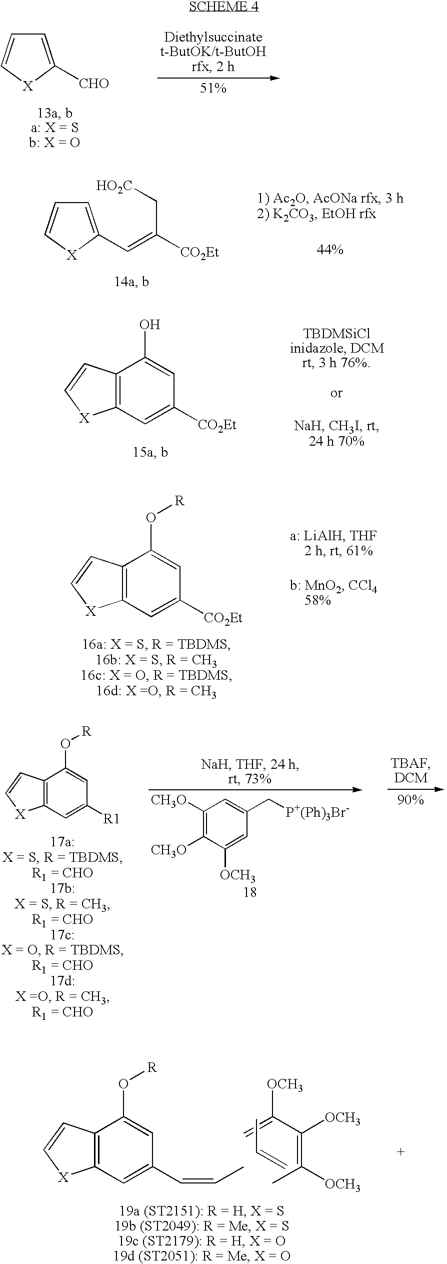 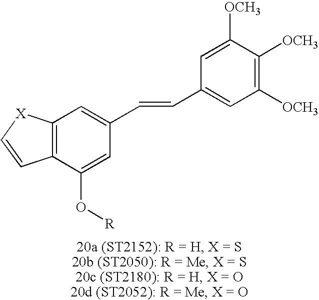 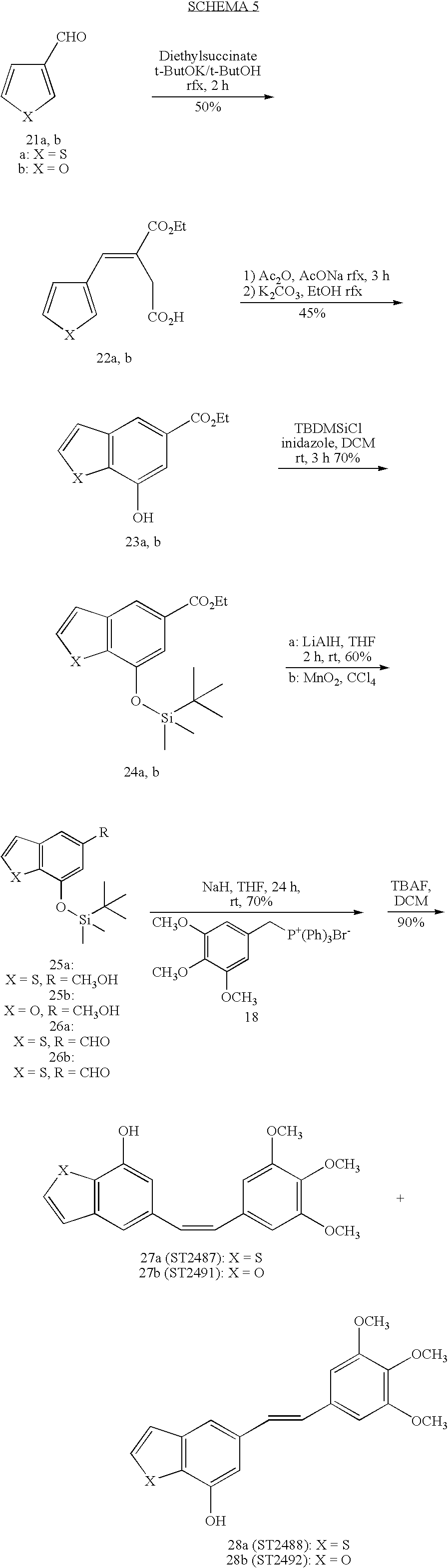
General Process for Obtaining 15a,b and 23a,b

To a suspension of t-BuOK (17 g.; 150 mmol, 3 equiv.) in t-BuOH (50 mL) is added a mixture of aldehyde 13a-b, 21a-b (50 mmol) in diethyl-succinate (32 mL, 225 mmol, 4.5 mmol). The reaction is refluxed for 45 minutes. After this time period the same amounts of t-BuOK, t-BuOH and diethyl-succinate are added and the mixture is left at reflux for another 45 minutes. It is then brought to room temperature, and acidified (pH 2) with an aqueous solution of HCl (20% v/v). The mixture is diluted with 5% HCl (100 mL) and extracted with EtOAc (3×100 mL). The organic phase is then extracted with 10% aqueous solution in Na₂CO₃ (4×50 mL); the pooled aqueous phases are washed with Et₂O (50 mL) and then acidified to pH=2 with HCl (20% v/v). The aqueous phase is finally extracted with EtOAc (4×50 mL) and the anhydrified pooled organic extracts are concentrated at reduced pressure, giving acid ester 14a-b, 22a-b with a quantitative yield. The crude product (14a-b, 22a-b) obtained with the previous reaction (50 mmol) is solubilised in a mixture consisting of acetic anhydride (100 mL) and anhydrous CH₃CO₂ Na (200 mmol, 4 equiv.). The solution thus obtained is brought to the boil for 5 hours, after which it is evaporated to dryness. The residue is extracted with an aqueous solution (75 mL) of Na₂CO₃ (15%) and extracted with EtOAc (3×50 mL). The pooled organic extracts are washed with brine (50 mL), anhydrified (Na₂SO₄) and purified by flash chromatography on silica gel.

A suspension of acetylderivative (10 mmol) and anhydrous K₂CO₃ (1.4 g., 10 mmol) in EtOH (20 mL) is refluxed for 18 hours, after which it is filtered and the filtrate evaporated to dryness. The residue is solubilised in water (20 mL), the aqueous phase is acidified (pH=2) with HCl (10% v/v) and then extracted with EtOAc (3×20 mL). The pooled organic extracts are anhydrified (Na₂SO₄), concentrated at reduced pressure and purified by flash chromatography on silica gel.

15a: brown solid, m.p.=134-136° C.; 15b: white solid, m.p.=105-107° C.; 23a: brown solid, m.p.=145-147° C.; 23b: white solid, m.p.=165-167° C.

Preparation of 16b, 16d

To a suspension consisting of compound 15a,b (5 mmol) and anhydrous K₂CO₃ (5 mmol, 690 mg, 1 equiv.) in THF (20 mL) is added Me₂SO₄ (5 mmol, 630 mg, 0.48 mL) and the resulting solution is brought to the boil for 8 h. After this period the mixture is filtered, evaporated to dryness and the residue extracted with a mixture of EtOAc (20 mL) and water (5 mL). The organic phase is washed with brine (5 mL), anhydrified and concentrated in vacuo. The resulting residue is purified by flash chromatography on silica gel. Derivatives 16b,d are obtained as colourless oils.

Preparation of 16a,c and 24a,b

To a solution of phenol 15a-b, 23a-b (3 mmol) in DCM (10 mL) are added TBDMSCl (3.6 mmol, 1.2 equiv., 550 mg) and imidazole (7.5 mmol, 2.5 equiv., 510 mg). The mixture is left at room temperature for 18 hours, after which it is diluted with DCM (10 mL), washed with water (5 mL) and brine (5 mL) and the organic phase is anhydrified. After concentration, the residue is purified by flash chromatography on silica gel. Derivatives 16a, 16c, 24a and 24b are obtained as colourless oils.

Preparation of 17a-d and 26a,b

The appropriate ester 16a-d, 24a,b (2 mmol) dissolved in THF (5 mL) is added dropwise at 0° C. to a suspension of LiAlH₄ (3 mmol, 114 mg, 1.5 equiv.) in 10 mL of THF. On completion of the addition, the reaction is left for a further 30 minutes at 0° C. and then for 2 h at room temperature. The reaction is then cooled again with a water and ice bath, the excess LiAlH₄ is decomposed with an aqueous soda solution (5%); the reaction mixture is filtered on celite, and the filtrate extracted with EtOAc (15 mL) and water (5 mL). The organic phase is then washed with brine (5 mL), anhydrified (Na₂SO₄) and evaporated to dryness. The product obtained is purified by flash chromatography on silica gel. To a solution of the alcohol derivative obtained by chromatography (1 mmol) in CCl₄ (25 mL) is added MnO₂ (1.1 mmol, 1.1 equiv.). After 2 h at room temperature, the mixture is filtered and the filtrate evaporated to dryness and used for the next reaction without any further purification.

Preparation of ST2151, ST2152, ST2179, ST2180, ST2049, ST2050, ST2051 and ST2052

To a solution of aldehyde 17a-d, 26a,b (2 mmol) in 10 mL of anhydrous THF is added phosphonium salt 18 (2 mmol, 1.05 g, 2 equiv.). The suspension thus obtained is cooled with a water and ice bath, and NaH (50% in mineral suspension, 2.2 mmol, 1.1 equiv., 110 mg) is then added. It is left to stir at room temperature for 24 hours and filtered on a celite bed, washing with THF. Evaporation is performed and the residue is extracted with DCM (15 mL), and the organic phase is washed with water (5 mL) and brine (5 mL), anhydrified and evaporated again.

For the derivatives in which the phenol oxyhydryl is protected as TBDMS ether, the residue is dissolved in DCM (10 mL), and TBAF (6 mmol, 3 equiv.) is added. After 1 hour at room temperature, the mixture is diluted with DCM (5 mL), washed with water (3×5 mL) and brine (5 mL) and anhydrified (Na₂SO₄). After concentration, the residue is purified with flash chromatography on silica gel.

For the purification of these products chromatography on silica gel is used with an elution gradient of the following type: EtOAc:petroleum ether 1:9, 2:8, 3:7.

### Cis-6-[2-(3,4,5-trimethoxy-phenyl)-vinyl]-benzo[b]thiophen-4-ol—ST2151

White solid, m.p.=145-147° C.;

¹H-NMR (CDCl₃)δ: 3.64 (s, 6H), 3.83 (s, 3H), 5.34 (s, 1H), 6.50 (d, J=12.6 Hz, 1H), 6.54 (s, 2H), 6.60 (d, J=12.6 Hz, 1H), 6.69 (s, 1H), 7.32 (d, J=5.6 Hz, 1H), 7.41 (d, J=5.6 Hz, 1H), 7.42 (s, 1H).

### Trans-6-[2-(3,4,5-trimethoxy-phenyl)-vinyl]-benzo[b]thio-phen-4-ol—ST2152

Yellow solid, m.p.=67-69° C.;

¹H-NMR (CDCl₃) δ: 3.88 (s, 6H), 3.92 (s, 3H), 5.50 (s, 1H), 6.74 (s, 2H), 6.93 (s, 1H), 7.03 (s, 2H), 7.35 (d, J=5.2 Hz, 1H), 7.43 (d, J=5.2 Hz, 1H), 7.56 (s, 1H).

### Cis-4-Methoxy-6-[2-(3,4,5-trimethoxy-phenyl)-vinyl]-benzo[b]thiophene—ST2049

Yellow oil.

¹H-NMR (CDCl₃) δ: 3.65 (s, 6H), 3.76 (s, 3H) 3.84 (s, 3H), 6.55 (s, 2H), 6.58 (d, J=11.2 Hz, 1H), 6.64 (d, J=11.2 Hz, 1H), 6.70 (s, 1H), 7.31 (d, J=5.0 Hz, 1H), 7.42 (d, J=5.0 Hz, 1H), 7.44 (s, 1H).

FAB-MS (MALDI-TOF): 356.4 [M+1].

### trans-4-Methoxy-6-[2-(3,4,5-trimethoxy-phenyl)-vinyl]-benzo[b]thiophene—ST2050

Yellow solid; m.p.=171-173° C.

¹H-NMR (CDCl₃) δ: 3.89 (s, 6H), 3.94 (s, 3H), 4.03 (s, 3H), 6.78 (s, 2H), 6.95 (s, 1H), 7.10 (s, 2H), 7.33 (d, J=5.6 Hz, 1H), 7.47 (d, J=5.6 Hz, 1H), 7.58 (s, 1H).

FAB-MS (MALDI-TOF): 356.3 [M+1].

### Cis-6-[2-(3,4,5-trimethoxy-phenyl)-vinyl]-benzofuran-4-ol—ST2179

White solid; m.p.=134-136° C.

¹H-NMR (CDCl₃) δ: 3.57 (s, 6H), 3.77 (s, 3H), 5.12 (s, 1H), 6.42 (d, J=12 Hz, 1H), 6.44 (s, 2H), 6.53 (d, J=12 Hz, 1H), 6.56 (s, 1H), 6.72 (d, J=2.2 Hz, 1H), 7.00 (s, 1H), 7.45 (d, J=2.2 Hz, 1H).

FAB-MS (MALDI-TOF): 327.2 [M+1].

### Trans-6-[2-(3,4,5-trimethoxy-phenyl)-vinyl]-benzofuran-4-ol—ST2180

Pale yellow solid, m.p.=142-143° C.

¹H-NMR (CDCl₃) δ: 3.89 (s, 6H), 3.92 (s, 3H), 5.50 (s, 1H), 6.74 (s, 2H), 6.93 (s, 1H), 7.03 (s, 2H), 7.35 (d, J=5.2 Hz, 1H), 7.43 (d, J=5.2 Hz, 1H), 7.56 (s, 1H).

### Cis-4-Methoxy-6-[2-(3,4,5-trimethoxy-phenyl)-vinyl]-benzo-furan—ST2051

Yellow oil.

¹H-NMR (CDCl₃) δ: 3.65 (s, 6H), 3.74 (s, 3H), 3.83 (s, 3H), 6.52 (s, 2H), 6.55 (d, J=11.2 Hz, 1H), 6.62 (d, J=11.2 Hz, 1H), 6.63 (s, 1H), 6.80 (s, 1H), 7.10 (s, 1H), 7.51 (s, 1H).

FAB-MS (MALDI-TOF): 356.4 [M+1].

### trans-4-Methoxy-6-[2-(3,4,5-trimethoxy-phenyl)-vinyl]-benzofuran—ST2052

Yellow solid, m.p.=152-153° C.

¹H-NMR (CDCl₃) δ: 3.88 (s, 6H), 3.94 (s, 3H), 4.00 (s, 3H), 6.76 (s, 2H), 6.84 (s, 2H), 7.08 (s, 2H), 7.28 (d, J=2.2 Hz, 1H), 7.54 (d, J=2.2 Hz, 1H).

FAB-MS (MALDI-TOF): 340.6 [M+1].

### Cis-5-[2-(3,4,5-trimethoxy-phenyl)-vinyl]-benzo[b]thiophen-7-ol—ST2487

Brown solid, m.p.=152-154° C.

¹H-NMR (CDCl₃) δ: 3.63 (s, 6H), 3.83 (s, 3H), 5.51 (s, 1H), 6.48 (d, J=12.2 Hz, 1H), 6.52 (s, 2H), 6.64 (d, J=12.2 Hz, 1H), 6.73 (s, 1H), 7.29 (d, J=3.2 Hz, 1H), 7.41 (d, J=3.2 Hz, 1H), 7.43 (s, 1H).

### trans-5-[2-(3,4,5-trimethoxy-phenyl)-vinyl]-benzo[b]thiophen-7-ol—ST2488

Pale yellow solid, m.p.=172-174° C.;

¹H-NMR (CDCl₃) δ: 3.89 (s, 6H), 3.92 (s, 3H), 5.63 (s, 1H), 6.74 (s, 2H), 6.94 (s, 1H), 7.02 (d, J=2.8 Hz, 1H), 7.32 (d, J=5.2 Hz, 1H), 7.45 (d, J=5.2 Hz, 1H), 7.53 (s, 1H).

### cis-5-[2-(3,4,5-trimethoxy-phenyl)-vinyl]-benzofuran-7-ol—ST2491 (27b)

White solid, m.p.=140-141° C.;

¹H-NMR (CDCl₃) δ: 3.63 (s, 6H), 3.83 (s, 3H), 5.20 (s, 1H), 6.46 (d, J=12.2 Hz, 1H), 6.52 (s, 2H), 6.57 (d, J=12.4 Hz, 1H), 6.69 (d, J=2.2 Hz, 1H), 6.82 (s, 1H), 7.12 (s, 1H), 7.58 (d, J=2.2 Hz, 1H).

### trans-5-[2-(3,4,5-trimethoxy-phenyl)-vinyl]-benzofuran-7-ol—ST2492 (28b)

White solid, m.p.=173-175° C.;

¹H-NMR (CDCl₃) δ: 3.89 (s, 6H), 3.92 (s, 3H), 6.01 (s, 1H), 6.74 (s, 2H), 6.97 (s, 1H), 7.06 (d, J=3.2 Hz, 1H), 7.26 (d, J=5.2 Hz, 1H), 7.45 (d, J=5.2 Hz, 1H), 7.60 (s, 1H).

By means of an analogue process were obtained:

### cis-6-[2-(3,5-dimethoxy-phenyl)-vinyl]-benzo[b]thiophen-4-ol—ST2900

¹H NMR δ (CDCl₃): 3.63 (s, 6H), 5.06 (s, 1H), 6.32-6.34 (m, 1H), 6.45 (d, J=2.2 Hz, 2H), 6.53 (d, J=12.4 Hz, 1H), 6.60-6.66 (m, 2H), 7.34 (s, 1H), 7.38-7.40 (m, 2H).

### cis-5-[2-(3,5-dimethoxy-phenyl)-vinyl]-benzofuran-7-ol—ST2901

¹H NMR δ (CDCl₃): 3.62 (s, 6H), 5.07 (s, 1H), 6.30-6.32 (m, 1H), 6.43 (d, J=2.2 Hz, 2H), 6.48 (d, J=12.2 Hz, 1H), 6.64 (d, J=12.2 Hz, 1H), 6.67.6.69 (m, 1H), 6.78 (d, J=1.4 Hz, 1H), 7.10 (s, 1H), 7.57 (d, J=2.2 Hz, 1H)

### cis-6-[2-(3,5-dimethoxy-phenyl)-vinyl]-benzofuran-4-ol—ST2902

¹H NMR δ (CDCl₃): 3.64 (s, 6H), 4.93 (s, 1H), 6.31-6.34 (m, 1H), 6.43 (d, J=2.4 Hz, 2H), 6.53 (d, J=12.2 Hz, 1H), 6.57 (s, 1H), 6.63 (d, J=12.2 Hz, 1H), 6.78 (dd, J=2.2 Hz, J=1 Hz, 1H), 7.05 (s, 1H), 7.51 (d, J=2.2 Hz, 1H).

### EXAMPLE 4

Preparation of ST2053, ST2054, ST2055, ST2056, ST2057, ST2058, ST2181 and ST2182

These compounds are prepared according to synthesis Scheme 6 here below:

Aldehydes 29a,b were prepared with a synthesis process in all respects similar to that used to prepare aldehydes 17a,d (Scheme 4).

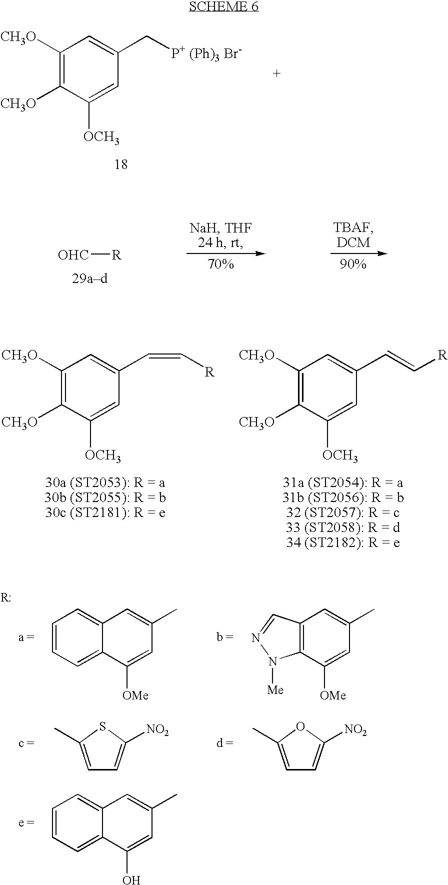

### cis-1-Methoxy-3-[2-(3,4,5-trimethoxy-phenyl)-vinyl]-naphthalene—ST2053

Colourless oil.

¹H-NMR (CDCl₃) δ: 3.63 (s, 6H), 3.75 (s, 3H), 3.83 (s, 3H), 6.57 (s, 1H), 6.66 (d, J=13.2 Hz, 1H), 6.71 (d, J=13.2 Hz, 1H), 6.75 (s, 1H), 7.44 (m, 4H), 7.69 (m, 1H), 8.12 (m, 1H).

FAB-MS (MALDI-TOF): 350.3 [M+1].

### trans-1-Methoxy-3-[2-(3,4,5-trimethoxy-phenyl)-vinyl]-naphthalene—ST2054

Yellow solid; m.p.=166-168° C.

¹H-NMR (CDCl₃) δ: 3.89 (s, 3H), 3.95 (s, 6H), 4.09 (s, 3H), 6.80 (s, 2H), 7.06 (s, 1H), 7.16 (s, 2H), 7.46 (m, 3H), 7.76 (dd, J=9.2 e 1.8 Hz, 1H), 8.20 (dd, J=9.2 e 1.8 Hz, 1H).

FAB-MS (MALDI-TOF): 350.3 [M+1].

### cis-7-Methoxy-1-methyl-5-[2-(3,4,5-trimethoxy-phenyl)-vinyl]-1H-indazole—ST2055

White solid, m.p. 182-183° C.;

¹H-NMR (CDCl₃) δ: 3.64 (s, 3H), 3.67 (s, 3H), 3.82 (s, 3H), 4.23 (s, 3H), 6.51 (d, J=12.5 Hz, 1H), 6.53 (s, 2H), 6.59 (d, J=12.5 Hz, 1H), 7.19 (s, 1H), 7.80 (s, 2H).

### trans-7-Methoxy-1-methyl-5-[2-(3,4,5-trimethoxy-phenyl)-vinyl]-1H-indazole—ST2056

Oil;

¹H-NMR (CDCl₃) δ: 3.86 (s, 3H), 3.91 (s, 6H), 4.01 (s, 3H), 4.28 (s, 3H), 6.73 (s, 2H), 6.94 (d, J=15.8 Hz, 1H), 7.06 (d, J=15.8 Hz, 1H), 7.31 (s, 1H), 7.86 (s, 2H).

### 2-Nitro-5-[2-(3,4,5-trimethoxy-phenyl)-vinyl]-thiophene—ST2057

Yellowish oil,

¹H NMR (CDCl₃) δ 3.89 (s, 3H), 3.93 (s, 6H), 6.73 (s, 2H), 6.99 (d, 1H, J=4.4 Hz), 7.06 (s, 2H), 7.85 (d, 1H, J=4.4 Hz).

### 2-Nitro-5-[2-(3,4,5-trimethoxy-phenyl)-vinyl]-furan—ST2058

Yellowish oil.

¹H NMR (CDCl₃) δ 3.90 (s, 3H), 3.92 (s, 6H), 6.53 (d, 1H, J=3.7 Hz), 6.76 (s, 2H) 7.28 (s, 2H), 7.38 (d, 1H, J=3.6 Hz).

### cis-3-[2-(3,4,5-trimethoxy-phenyl)-vinyl]-naphthalen-1-ol—ST2181

Yellow solid, m.p.=163-165° C.

¹H-NMR (CDCl₃) δ: 3.62 (s, 6H), 3.84 (s, 3H), 5.56 (s, 1H), 6.53 (d, J=12.4 Hz, 1H), 6.56 (s, 2H), 6.68 (d, J=12.4 Hz, 1H), 6.79 (s, 1H), 7.38 (s, 1H), 7.45 (m, 2H), 7.72 (dd, J=9.8 e 3.6 Hz, 1H), 8.11 (dd, J=9.8 e 3.6 Hz, 1H).

### trans-3-[2-(3,4,5-trimethoxy-phenyl)-vinyl]-naphthlen-1-ol—ST 2182

Yellow solid, m.p.=176-178° C.

¹H-NMR (CDCl₃) δ: 3.90 (s, 3H), 3.93 (s, 6H), 5.73 (s, 1H), 6.77 (s, 2H), 7.07 (m, 3H), 7.46 (m, 3H), 7.80 (dd, J=9.6 e 2.8 Hz, 1H), 8.14 (dd, J=9.6 e 2.8 Hz, 1H).

### EXAMPLE 5

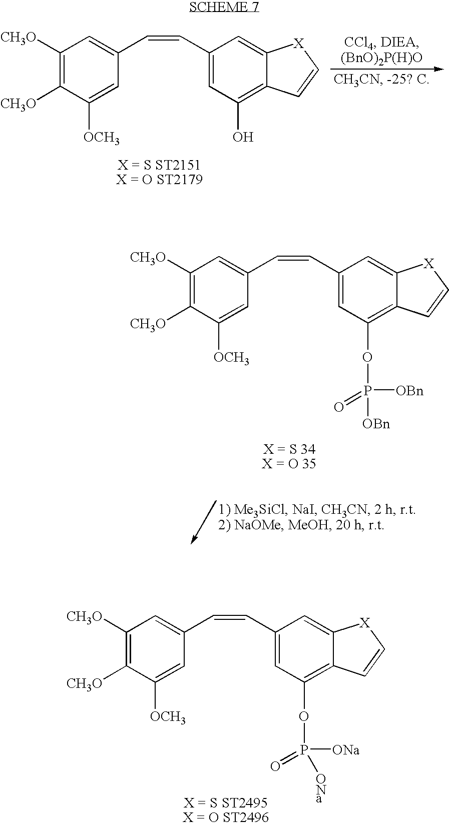 General Process for Obtaining 34 and 35

To a solution of 1.2 mmol of ST2151 (or ST2179) in 5 mL of anhydrous CH₃CN, cooled to −25° C., were added 581 μL (6 mmol; 5 eq.) of CCl₄. After approximately 10 minutes the following were added in the order indicated: 429 μL (2.59 mmol; 2.1 eq.) of diisopropylethylamine, 15 mg (0.12 mmol; 0.1 eq.) of dimethylaminopyridine and 383 μL (1.74 mmol; 1.45 eq.) of dibenzyl-phosphyte. After 2 h at −10° C. the reaction was complete and was added with 20 mL of KH₂PO₄ 0.5 M, and the aqueous phase was shaken with AcOEt (3×10 mL). The organic phases were dried on anhydrous Na₂SO₄ and the crude product was purified by chromatography on SiO₂ with hexane:AcOEt 75:25 to give 1.05 mmol: yield: 88% of the expected product as a yellow oil.

### 6[(Z)-2-(3,4,5-trimethoxyphenyl)ethenyl]-1-benzothiophen-4-olo 4-O-dibenzyl-phosphate (34)

Fr=0.11 in hexane/AcOEt 8:2, MS-IS:M+H]⁺=603.2

¹H-NMR (300 MHz, CDCl₃) δ: 3.6 (s, 6H, 2×OCH₃), 3.8 (s, 3H, OCH₃), 5.05 (s, 2H, CH₂), 5.1 (s, 2H, CH₂), 6.5 (s, 2H, 2×CHar), 6.6 (bs, 2H, 2×CHar), 7.2-7.4 (m, 11H, 11×CHar), 7.6 (s, 1H, CHar).

¹³C-NMR (75 MHz, CDCl₃) δ: 56.1; 61.1; 70.3; 106.4; 115.9; 119.7; 120.4; 127.1; 128.2; 128.8; 128.9; 129.0; 131.1; 131.6; 132.2; 134.9; 135.6; 153.2.

### 6[(Z)-2-(3,4,5-trimethoxyphenyl)ethenyl]-1-benzofuran-4-ol-4-O-dibenzyl-phosphate (35)

Fr=0.20 in hexane/AcOEt 7:3, MS-IS:M+H]⁺=587.2

¹H-NMR (300 MHz, CDCl₃) δ: 3.6 (s, 6H, 2×OCH₃), 3.8 (s, 3H, OCH₃), 5.05 (s, 2H, CH₂), 5.1 (s, 2H, CH₂), 6.45 (s, 2H, 2×CHar), 6.55 (bs, 2H, 2×CHar), 6.75 (bs, 1H, CHar), 7.05 (s, 1H, CHar), 7.2-7.4 (m, 11H, 11×CHar), 7.5 (bs, 1H, CHar).

¹³C-NMR (75 MHz, CDCl₃) δ: 56.1; 61.1; 70.3; 98.8; 104.3; 106.4; 109.1; 115.1; 119.9; 128.2; 128.8; 128.9; 129.2; 130.9; 132.3; 134.7; 135.5; 145.5; 153.2; 156.5.

General Process for Obtaining ST2495 and ST2496

To the solution of 1.2 mmol of dibenzyl-ester 34 (or 35) in 7 mL of anhydrous CH₃CN were added, at room temperature, 36 mg (2.4 mmol; 2 eq.) of NaI and then the solution of 303 μL (2.4 mmol; 2 eq.) of Me₃SiCl in 1 mL of anhydrous CH₃CN. After 2 h the reaction was complete and the minimum amount of water to solubilise the salts was added, as well as a 10% Na₂S₂O₃ solution until decoloration of the reaction mixture was achieved. The solution thus obtained was shaken with AcOEt until complete extraction of the product in the organic phase; the organic phases were dried on Na₂SO₄ and the solvent removed in vacuo.

The benzyl-ester can be removed by BTMS [S. Lazar, et al., Synthetic Comm. 1992, 22(6), 923-31] but the reaction was less rapid than with NaI.

The crude oil thus obtained was dissolved in 4 mL of anhydrous MeOH, and 130 mg (2.4 mmol; 2 eq.) of NaOMe were added to the solution. The mixture was left at room temperature for 20 h, until complete salification was achieved. The solvent was then removed in vacuo and the residue washed with Et₂O to give 1.1 mmol (yield: 92%) of product as a white solid.

Alternatively, the sodium salt can be prepared in NaOH 1N solution.

### Disodium 6[(Z)-2-(3,4,5-trimethoxyphenyl)ethenyl]-1-benzo-thiophen-4-ol-4-O-phospohate—ST2495

T dec=226°, MS-IS:[M−1]⁺=419.

¹H-NMR (300 MHz, D₂O) δ: 3.4 (s, 3H, OCH₃), 3.1 (s, 3H, OCH₃), 3.75 (s, 3H, OCH₃), 6.4-6.45 (d, 1H, CHolef), 6.5(s, 2H, 2×CHar), 6.1-6.15 (d, 1H, CHolef), 7.25-7.5 (m, 4H, 4×CHar).

¹³C-NMR (75 MHz, D₂O) δ: 30.4; 55.7; 56.0; 56.2; 61.1; 104.1; 106.9; 115.5; 116.6; 121.5; 121.6; 126.1; 126.3; 127.6; 128.3; 128.6; 128.9; 129.9; 130.7; 132.6; 132.7; 133.6; 134.3; 134.7; 136.1; 140.9; 141.6; 149.2; 152.3; 152.8.

### Disodium 6[(Z)-2-(3,4,5-trimethoxyphenyl)ethenyl]-1-benzo-furan-4-ol 4-O-phosphate—ST2496

T dec=2120, MS-IS:[M−1]⁺=403.

¹H-NMR (300 MHz, D₂O) δ: 3.5 (s, 6H, 2×OCH₃), 3.6 (s, 3H, OCH₃), 6.4-6.45 (d, 1H, CHolef), 6.5(s, 2H, 2×CHar), 6.6-6.65 (d, 1H, CHolef), 6.85-7.1 (m, 3H, 3×CHar), 7.5 (s, 1H, CHar).

¹³C-NMR (75 MHz, D₂O) δ: 30.4; 55.8; 56.0; 56.2; 61.1; 98.8; 104.1; 104.2; 104.8; 105.9; 106.8; 114.9; 120.3; 127.6; 128.0; 128.6; 129.7; 130.9; 133.7; 134.2; 136.1; 145.2; 147.4; 152.1; 152.3; 152.8; 156.0.

Also objects of the present invention are the intermediate synthesis products 15a,b, 16a-d, 17a-d, 23a,b, 24a,b, and 26a,b described in Schemes 4 and 5.

Preparation of 43(ST2898), 44, 45a,b (ST2899, ST2897), and 46a,b

The compounds are pre-pared according to synthesis in Schemes 8 and 9 here below 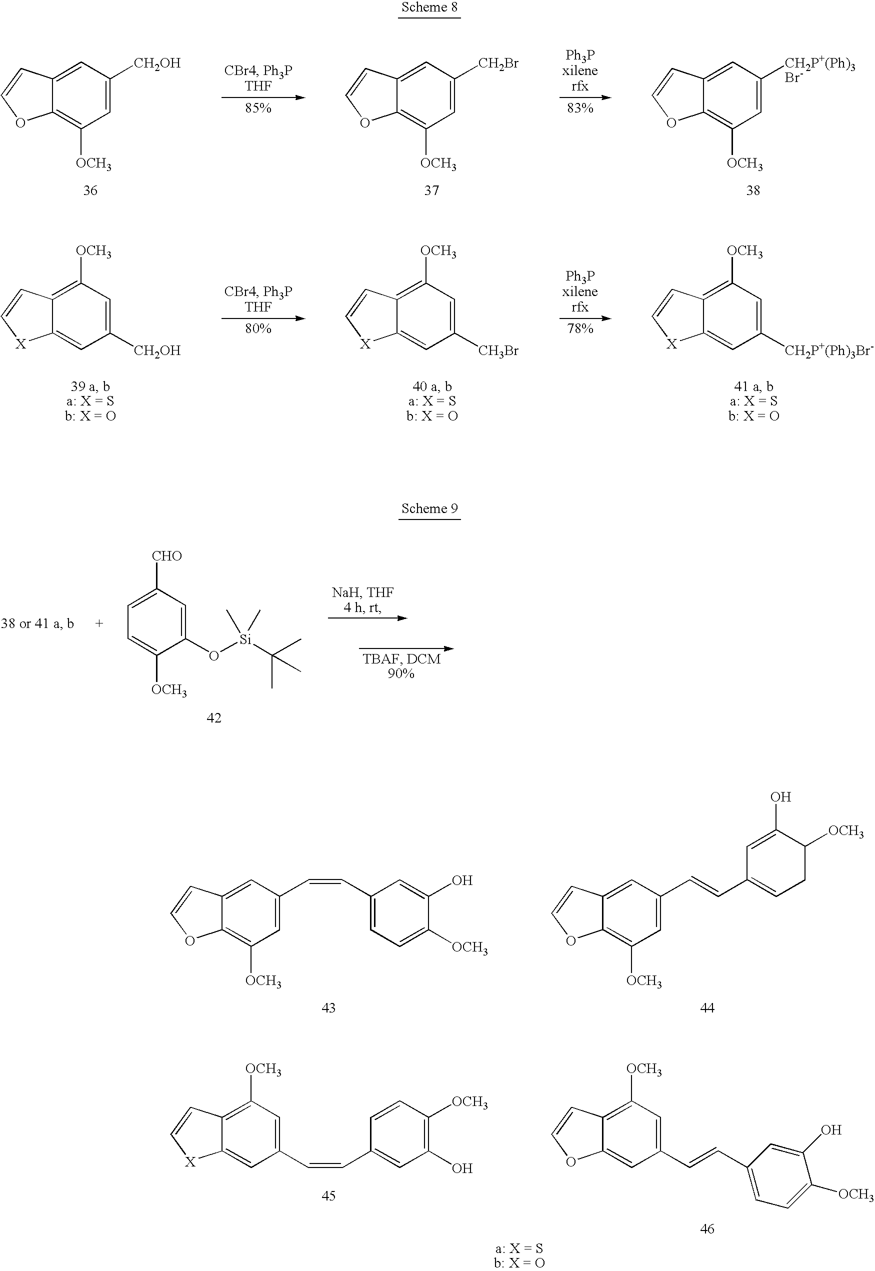
General Process for Obtaining 37 and 40

To the appropriate alcohol 36, or 39 a,b (1.8 mmol) dissolved in THF (15 mL) is added CBr₄ (2.87 mmol, 953 mg, 1.6 equiv.) and P(Ph)₃ 2.87 mmol, 754 mg, 1.6 equiv.) at 0° C. The reaction is left at room temperature for 1.5 hours; after which the mixture is diluted with EtOAc (10 mL), washed with water (5 mL) and brine (5 mL) and the organic phase is dried. After concentration, the residue is purified by flash chromatography on silica gel. The derivatives 37 and 40a,b are obtained as colourless oils.

### 5-Bromomethyl-7-methoxy-benzofuran

Yield: 54%. Oil.

¹HNMR (CDCl₃) δ 4.03 (s, 3H), 4.61 (s, 2H), 6.74 (d, 1H, J=2.2 Hz), 6.84 (d, 1H, J=1.6 Hz), 7.23 (d, 1H, J=1.4), 7.64 (d, 1H, J=1.8).

### 6-Bromomethyl-4-methoxy-benzofuran

Yield: 80%. Oil.

¹HNMR (CDCl₃) δ 3.96 (s, 3H), 4.62 (s, 2H), 6.69 (d, 1H, J=1.2 Hz), 6.83-6.84 (m, 1H), 7.18 (s, 1H), 7.5 (d, 1H, J=2.2 Hz).

### 6-Bromomethyl-4-methoxy-benzothiophene

Yield: 60%. Oil.

¹HNMR (CDCl₃) δ 3.97 (s, 3H), 4.63 (s, 2H), 6.69 (d, 1H, J=1.3 Hz), 6.89 (s, 1H), 7.22(s, 1H), 7.59 (s, 1H).

General Process for Obtaining 38 and 41

To a solution of 37 or 40 a,b (0.97 mmol) in xilene (10 ml) is added P(Ph)₃ (1.65 mmol, 433 mg, 1.7 equiv.), and the resulting suspension is brought to the boil for 12 hours. The suspension is filtered, the residue is washed with diethyl ether and crystallized from methanol-diethyl ether. The salts 38 and 41 a,b are obtained as colourless solid materials with a m.p. over 180° C. with decomposition.

### 7-Methoxy-benzofuran-5-ylmethyl)-triphenyl-phosphonium

Yield: 85%, ¹H-NMR (CDCl₃) δ: 3.66 (s, 3H), 5.50 (d, 2H, J=13.6 Hz), 6.55 (d, 1H, J=2 Hz), 6.78 (s, 1H), 6.93 (s, 1H), 7.54-7.81 (m, 16H).

### 4-Methoxy-benzofuran-6-ylmethyl)-triphenyl-phosphonium

Yield: 83%.

¹H-NMR (CDCl₃) δ: 3.63 (s, 3H), 5.49 (d, 2H, J=14.2 Hz), 6.74-6.78 (m, 3H), 7.57-7.67 (m, 16H).

### 4-Methoxy-benzo[b]thiophen-6-ylmethyl-triphenyl-phosphonium

Yield: 80%, ¹H-NMR (CDCl₃) δ: 3.65 (s, 3H), 5.48 (d, 2H, J=14.1 Hz), 6.75-6.79 (m, 3H), 7.58-7.68 (m, 16H).

General Process for Obtaining 43 (ST2898), 44, 45 a,b (ST2899, ST2897) and 46 a,b

To a solution of the aldehyde 42, (359.6 mg, 1.35 mmol) in 10 mL of anhydrous THF is added the phosphonic salt 38, or 41 a,b (1.35 mmol, 1 equiv.). The suspension thus obtained is cooled in an ice bath, after which NaH is added (50% in mineral suspension, 1.62 mmol, 1.2 equiv., 77 mg). The reaction is left to stir at room temperature for 2 hours, then is filtered on a celite bed and is washed with THF. Evaporation is done and the residue is extracted with DCM (15 mL) and is washed with water (5 mL) and brine (5 mL), and then dried and evaporated again. The residue is dissolved in DCM (10 mL) and TBAF (6 mmol, 3 equiv.) is added. After 1 hour at room temperature, the solution is diluted with DCM (5 mL), washed with water (3×5 mL) and brine (5 mL) and then dried (Na₂SO₄). After concentration, the residue is purified by flash chromatography on silica gel using EtOAc-petroleum ether 2-8.

### Cis-2-Methoxy-5-[2-(7-methoxy-benzofuran-5-yl)-vinyl]-phenol ST2898

Oil.

¹H-NMR (CDCl₃)δ: 3.81 (s, 3H), 3.86 (s, 3H), 5.47 (s, 1H), 6.48 (d, J=12 Hz, 1H), 6.60 (d, J=12.2 Hz, 1H), 6.68 (d, J=2 Hz, 1H), 6.72 (s, 1H), 6.75-6.76 (m, 2H), 6.88 (d, J=2 Hz, 1H), 7.1 (s, 1H), 7.57 (d, J=2 Hz, 1H).

### Trans-2-Methoxy-5-[2-(7-methoxy-benzofuran-5-yl)-vinyl]-phenol

Oil.

¹H-NMR (CDCl₃)δ: 3.92 (s, 3H), 4.1 (s, 3H), 5.62 (s, 1H), 6.75 (d, J=1.8 Hz, 1H), 6.83-6.86 (m, 2H), 6.95-7.02 (m, 4H), 7.2 (s, 1H), 7.61 (d, J=2 Hz, 1H).

### Cis-2-Methoxy-5-[2-(4-methoxy-benzofuran-6-yl)-vinyl]-phenol ST2897

Oil.

¹H-NMR (CDCl₃)δ: 3.76 (s, 3H), 3.86 (s, 3H), 5.52 (br, 1H), 6.50 (d, J=12 Hz, 1H), 6.56-6.64 (m, 2H), 6.73 (s, 1H), 6.78-6.80 (m, 2H), 6.91 (d, J=2 Hz, 1H), 7.01 (s, 1H), 7.49 (d, J=2 Hz, 1H).

### Trans-2-Methoxy-5-[2-(4-methoxy-benzofuran-6-yl)-vinyl]-phenol

Oil.

¹H-NMR (CDCl₃)δ: 3.85 (s, 3H), 3.92 (s, 3H), 5.53 (s, 1H), 6.75-6.77 (m, 3H), 6.92 (d, J=2, 1H), 6.96 (s, 3H), 7.1 (d, J=2.2 Hz, 1H), 7.45 (d, J=2.2 Hz, 1H).

### Cis-2-Methoxy-5-[2-(4-methoxy-benzo[b]thiophen-6-yl)-vinyl]-phenol ST2899

Oil.

¹H-NMR (CDCl₃)δ: 3.74 (s, 3H), 3.87 (s, 3H), 5.50 (s, 1H), 6.52 (d, J=12 Hz, 1H), 6.60 (d, J=12. Hz, 1H), 6.68-6.72 (m, 2H), 6.78 (d, J=2 Hz, 1H), 6.91 (d, J=2 Hz, 1H), 7.29 (d, J=5.4 Hz, 1H), 7.37-7.44 (m, 2H).

### Trans-2-Methoxy-5-[2-(4-methoxy-benzo[b]thiophen-6-yl)-vinyl]-phenol

Oil.

¹H-NMR (CDCl₃)δ: 3.85 (s, 3H), 3.95 (s, 3H), 5.54 (s, 1H), 6.76-6.85 (m, 3H), 6.98 (s, 2H), 7.10 (d, J=2 Hz, 1H), 7.19(s, 1H), 7.36-7.39 (m, 1H), 7.47 (s, 1H).

Preparation of 19a (ST2151) and 19c (ST2179) by Photochemical Isomerization.

The compounds are prepared according to synthesis in Scheme 10 here below 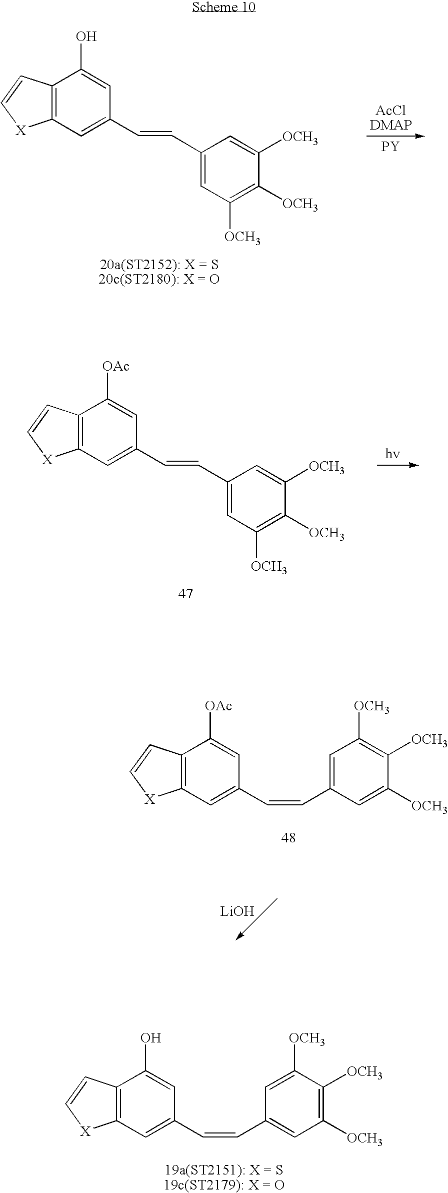
General Process for Obtaining 47a,c

To a solution of 20a (ST2152) or 20c (ST2180) (1,2 mmoli) in dichlorometane (4,8 ml), pyridine (2.1 eq.) and 4-dimethylaminopyridine (catalytic amount) were added and stirred in a dry flask.

Acetyl chloride (2 eq.) solubilized in dichlorometane (1,9 ml) was added dropwise at 0° C. and the mixture was stirred overnight at rt. The reaction was diluted with dichlorometane, and washed two times with HCl (acq. sol. 10%), twice with water, twice with saturated bicarbonate solution and once with saturated brine. The organic layer was dried over anhydrous sodium sulphate and concentrated in vacuo. The crude material was used without further purification.

General Process for Obtaining 48a,c

The crude 47 was divided in 300 mg portions and solubilized in methanol (300 ml ca.), all portions were treated as follows: the UV-VIS lamp was soaked in the solution and turned on. After about 45 minutes the light was switched off, the lamp was taken out and solvent was evacuated. The resulting crude materials were combined and used without further purification.

General Process for 19a (ST2151)

To a stirred solution of 48 (1 mmoli) in THF (3 ml), methanol (1 ml) and water (1 ml) was added lithium hydroxide (3 eq.) at 0° C. After 1 hour at rt the mixture was concentrated in vacuo, diluted with water and washed with diethyl ether (two times). The aqueous layer was acidified, extracted with diethyl ether (three times) and dried over anhydrous sodium sulphate. The product was purified with a silica gel column chromatography. The total yield starting from 20a,c is about 50%.

Photochemical isomerization: Both drug and prodrug forms of the stilbene derivatives, object of this patent, could be photoisomerizated by exposure to electromagnetic radiation, especially ultraviolet-visible light. In organic solution (MeOH, AcOEt, etc.), under argon, independently of E/Z starting isomer, there is a photochemical isomerization with, usually, an E/Z ratio of 70:30.

General Process for Obtaining Compounds 54 and 55.

These compounds can be obtained in the same manner as described for the ST2151 and ST2179 (Scheme 11). 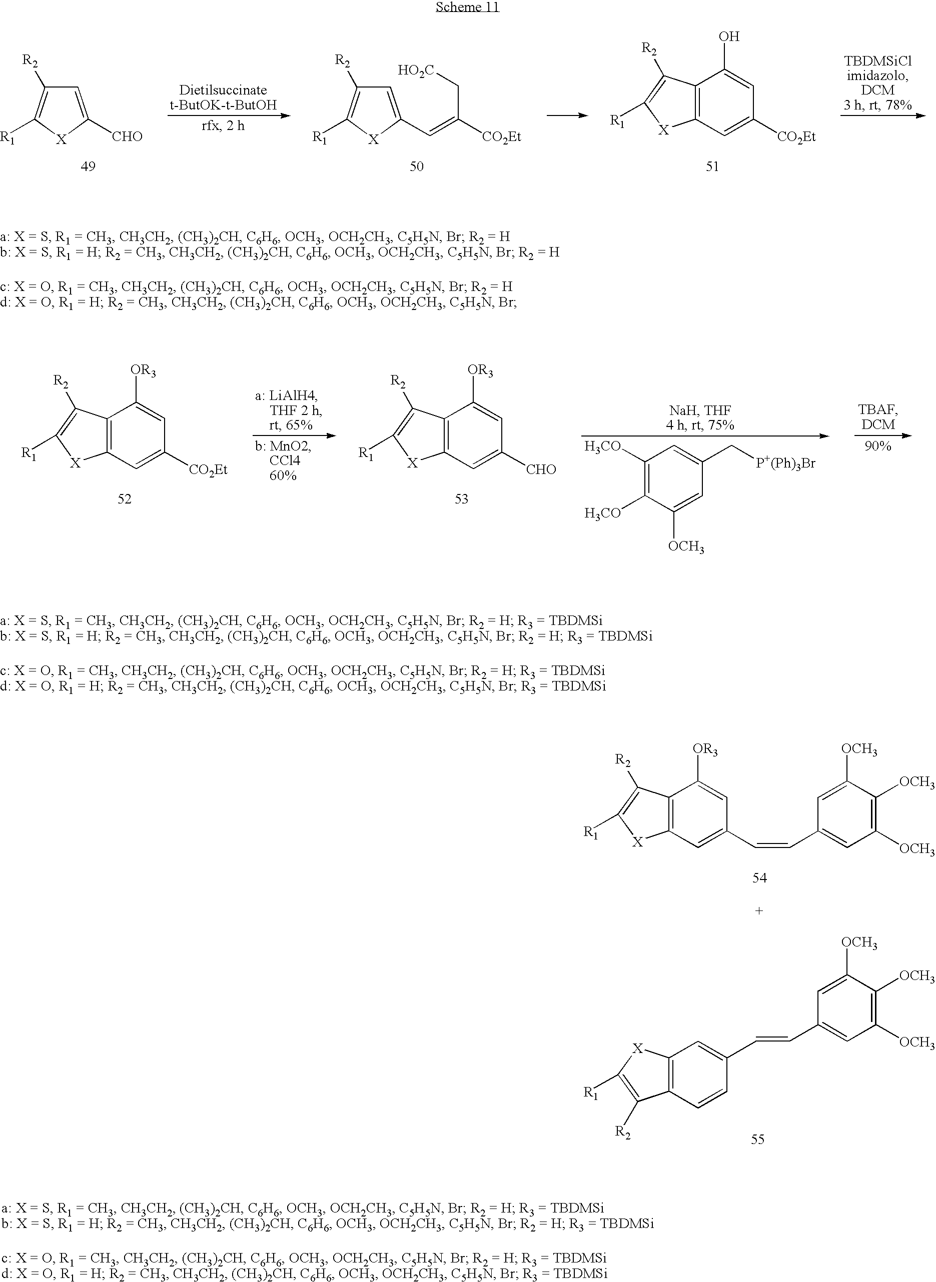
General Process for Obtaining Compounds 56.

The prodrug 56 was prepared by the route described in Scheme 12.

Typical methyloxy-phosphorylation method was first treated the phenolic residue with sodium hydride followed by protected chloromethyl phosphate prepared as a described method Mantyla A. et al. Tetrahedron Lett. 2002, 43, 3793-4). The protecting group was removal by a saturated EtOAc/HCl solution, followed by a disodium salt preparation in NaOH/H₂O solution. 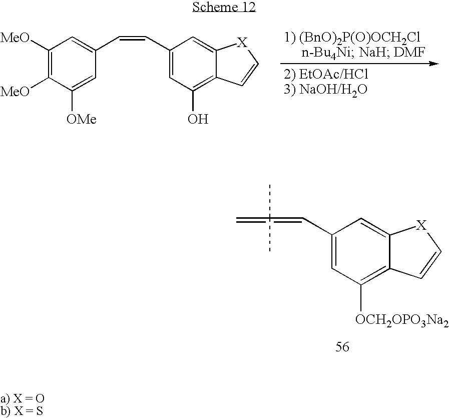
General Process for Obtaining Compounds 57.

Starting with aminostilbene derivatives, the coupling with aminoacids has been produced by Fmoc route, followed by cleavage of the α-amino protecting group [G. R. Pettit et al., J. Med. Chem 2002, 46, 525-31] 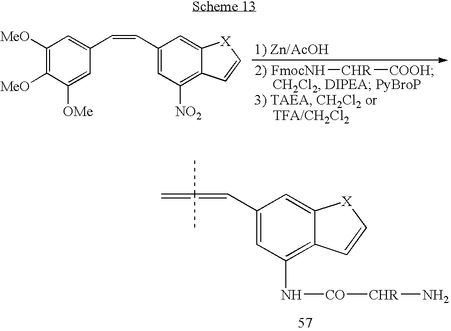 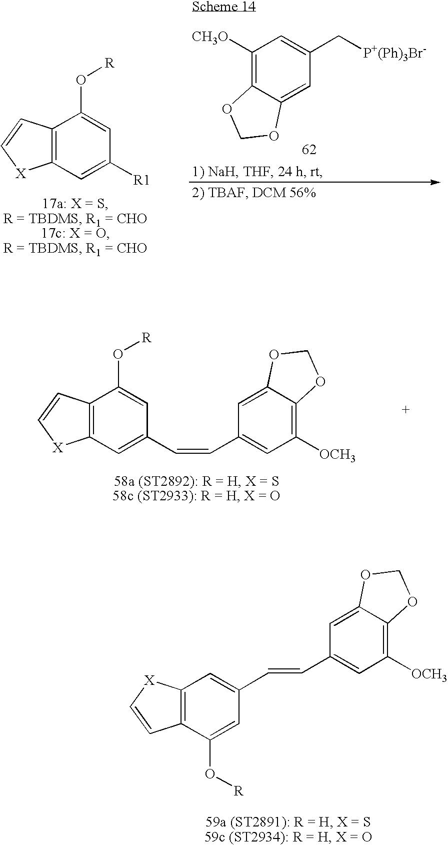
General Process for Obtaining ST2891, ST2892, Compounds ST2933 and ST2934

To a suspension of NaH (80% in mineral suspension, 7.4 mmol, 3.7 equiv., 220 mg) phosphonium salt (62) (8 mmol, 4.06 g., 4 equiv.) is added and the mixture is stirred for 30 min. at rt. A solution of aldehyde 17a-c (2 mmol) in 10 mL of anhydrous THF is then added to the reaction mixture, previously cooled down to 4° C. It is left to stir at room temperature for 1.5 hours and filtered on a celite bed, washing with THF. Evaporation is performed and the residue is extracted with DCM (15 mL), and the organic phase is washed with water (5 mL) and brine (5 mL), anhydrified and evaporated again.

For the derivatives in which the phenol oxyhydryl is protected as TBDMS ether, the residue is dissolved in DCM (10 mL), and TBAF (6 mmol, 3 equiv.) is added. After 1 hour at room temperature, the mixture is diluted with DCM (5 mL), washed with water (3×5 mL) and brine (5 mL) and anhydrified (Na₂SO₄). For the purification of these products chromatography on silica gel is used with an elution gradient of the following type: Hexane/EtOAc 99:1, 9:1, 85:15.

### 6-[(Z)-2-(7-methoxy-1,3-benzodioxol-5-yl)vinyl]-1-benzothiophene-4-ol—ST2892;

White solid, m.p.=121-123° C.

¹H-NMR (CDCl₃) δ: 3.68 (s, 3H), 5.93 (s, 2H), 6.49 (d, J=12.3 Hz, 1H), 6.51 (s, 2H), 6.56 (d, J=12.3 Hz, 1H), 6.67 (s, 1H), 7.33 (d, J=5.5 Hz, 1H), 7.39 (s, 1H), 7.40 (d, J=5.5 Hz, 1H).

### 6-[(E)-2-(7-methoxy-1,3-benzodioxol-5-yl)vinyl]-1-benzothiophene-4-ol—ST2891

White solid, m.p.=148-150° C.

¹H-NMR (CDCl₃) δ: 3.95 (s, 3H), 5.99 (s, 2H), 6.66 (s, 1H), 6.76 (s, 1H), 6.90 (s, 1H), 6.98 (s, 2H), 7.34 (d, J=5.5 Hz, 1H), 7.42 (d, J=5.5 Hz, 1H), 7.53 (s, 1H).

### 6-[(Z)-2-(7-methoxy-1,3-benzodioxol-5-yl)vinyl]-1-benzothiophene-4-ol—ST2933

Pale yellow oil.

¹H-NMR (CDCl₃) δ: 3.72 (s, 3H), 5.95 (s, 2H), 6.48 (d, J=9.3 Hz, 1H), 6.49 (s, 2H), 6.53 (d, J=9.3 Hz, 1H), 6.61 (s, 1H), 6.81 (d, J=2.2 Hz, 1H), 7.07 (s, 1H), 7.54 (d, J=2.2 Hz, 1H).

MS=309 [M−1]

### 6-[(E)-2-(7-methoxy-1,3-benzodioxol-5-yl)vinyl]-1-benzothiophene-4-ol—ST2934

White solid.

¹H-NMR (CDCl₃) δ: 3.96 (s, 3H), 6.01 (s, 2H), 6.66 (d, J=1.5 Hz, 1H), 6.76 (d, J=1.5 Hz, 1H), 6.85 (s, 1H), 6.86 (d, J=2.2 Hz, 1H), 6.97 (s, 2H), 7.23 (s, 1H), 7.56 (d, J=2.2 Hz, 1H). MS=309 [M−1] 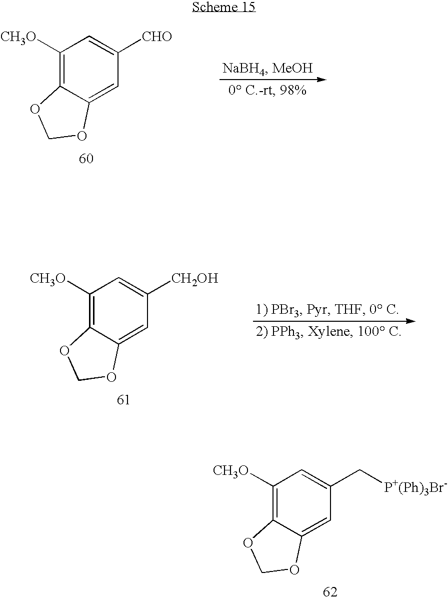
General Process for Obtaining 61

NaBH₄ (65.4 mmol, 1.2 eq., 2.47 g) is added, at 4° C., to a solution of compound 60 (54.5 mmol, 9.82 g). The reaction is complete in 1 hour, the solvent has been removed under reduced pressure, the residue has then been washed between EtOAc and water and the crude product purified by silica gel chromatography using the following gradient: Hexane/EtOAc 9:1, 7:3, 65:35.

White solid, m.p.=64-66° C.

¹H-NMR (CDCl₃) δ: 2.24 (s, 1H), 3.92 (s, 3H), 4.58 (s, 2H), 5.98 (s, 2H), 6.56 (s, 2H).

General Process for Obtaining 62

To a solution of 61 (53.8 mmol, 9.8 g) and pyridine (0.260 mL) in THF (100 mL), PBr₃ (134.5 mmol, 12 mL, 2.5 eq.) has been added at 4° C. After three hours at this temperature the reaction is complete and the reaction mixture has been washed between water and diethyl ether, the collected organic layers have been neutralized with saturated Na—HCO₃ and anhydrified over Na₂SO₄. The solvent has been removed under reduced pressure.

A solution of PPh₃ (64.5 mmol, 16.93 g, 1.2 eq.) in xylene (100 mL) has been dropped into a solution of the crude bromide in xylene (120 mL); the reaction mixture has been refluxed for 1.5 hours until completion. The suspension has been cooled down to room temperature and filtered to obtain the desired product as a white solid.

White solid, m.p.=159-162° C.

¹H-NMR (CD₃OD) δ: 3.56 (s, 3H), 5.93 (s, 2H), 6.17-6.22 (m 2H), 7.64-7.98 (m, 17H).

Cell Cultures and Cytotoxicity Tests

The cytotoxic effect of our derivatives was evaluated in series of human and murine cell lines.

Human umbilical vein endothelial cells (HUVEC), from the BioWhittaker company, were maintained in EGM-2 culture medium (BioWhittaker).

Bovine microcirculatory endothelial cells (BMEC), isolated from bovine adrenal glands, were maintained in culture in DMEM containing 20% FBS, 50 μg/ml of bovine brain extract (BBE), 50 units/ml of heparin (SIGMA), 100 units/ml of gentami-cin (SIGMA) and 10 mg/ml of L-glutamine (Hyclone). EA-hy926, an immortalised hybridoma of HUVEC and adenocarcinoma cells, obtained from the University of Bari Department of Biomedical Sciences and Human Oncology, was cultured in DMEM added with 10% FBS and gentamicin.

The following cell lines, purchased from ATCC, were cultured according to the manufacturer's instructions: MeWo human melanoma, NCI-H460 human lung cancer, LoVo human colon adenocarcinoma, PC3 human prostate carcinoma, MES-SA human uterine sarcoma, HCT 116 human colorectal carcinoma, MCF-7 human breast carcinoma.

The M109 murine lung cancer line, the HT29 human colon adenocarcinoma line, the A2780 human ovarian carcinoma line, obtained from the Milan Tumour Institute, were cultured in RPMI containing 10% FBS and antibiotics.

The B16/BL6 murine melanoma line, obtained from the M. Negri Institute in Milan, was cultured in DMEM containing 10% FBS and antibiotics.

For the cytotoxicity test the cells were seeded at variable densities according to cell type in 96-well plates in normal culture medium (200 μl/well) and incubated for 24 hours at 37° C. On the next day, the study substances were added at scalar concentrations and the cells were incubated for a further 24 hours at 37° C. in a humidified atmosphere containing 5% CO₂. At the end of the incubation period the medium containing the substances was removed and three washings with PBS were performed. At the end of the washings 200 μl/well of fresh medium were added and the plates were incubated at 37° C. for a further 48 hours. At the end of this incubation period the culture medium was removed by overturning the plates and 200 μl/well of PBS and 50 μl of 80% cold trichloroacetic acid (TCA) were added. The plates were then incubated in ice. After 1 h the TCA was removed, the plates were washed three times by immersion in distilled water and dried first on blotting paper and then in the oven. 200 μl of 0.4% sulforodamine B in 1% acetic acid were then added to all wells. The plates were incubated at room temperature for a further 30 minutes. The sulforodamine B was removed by overturning, the plates were washed three times by immersion in 1% acetic acid, and then dried first on blotting paper and then in the oven. 200 μl of Tris base 10 mM were then added to all wells and the plates were placed under stirring for at least 20 min. The optical density was measured by spectrophotometric readout at 540 nm.

Table 1 shows the IC₅₀ values of ST2151 and ST2179, that is to say the concentration capable of inhibiting cell survival by 50%, processed using ALLFIT software. In the same table are reported the IC₅₀ of ST2897, ST2898 and ST2899 on BMEC.

TABLE 1

| | IC₅₀ ± SE (nM) | | | |
|---|---|---|---|---|
| Cell line | ST2151 | ST2179 | ST2495 | ST2496 |
| BMEC | 87 ± 1 | 49 ± 1 | 640 ± 40 | 340 ± 16 |
| HUVEC | 49 ± 0.64 | n.d. | 83 ± 2.06 | 85 ± 1.2 |
| EAHY.926 | 52 ± 4.9 | 40 ± 3.9 | — | — |
| NCI-H460 | 74 ± 2.9 | 53 ± 1.3 | 620 ± 65 | 780 ± 3.4 |
| M109 | 490 ± 30 | 93 ± 6 | — | — |
| HT29 | 900 ± 65 | 990 ± 40 | >10000 | 3900 ± 70 |
| LoVo | 360 ± 0.01 | 490 ± 0.04 | — | — |
| PC3 | 120 ± 0.01 | 100 ± 0.01 | — | — |
| B16/BL6 | 85 ± 0.5 | 44 ± 3.8 | >10000 | 4140 ± 490 |
| A2780 | 70 ± 2 | 50 ± 2 | 500 ± 20 | 260 ± 6 |
| MeWo | 68 ± 5 | 71 ± 17 | 830 ± 0.13 | 820 ± 7.7 |
| MESSA | 86 ± 10 | 40 ± 4 | 1420 ± 49 | 500 ± 50 |
| HCT-116 | 84 ± 3.8 | 54 ± 9 | 3000 ± 102 | 1680 ± 70 |
| MCF-7 | 66 ± 2.7 | 38 ± 3 | 693 ± 21 | 646 ± 29 |

| | IC₅₀ ± SE (nM) | | |
|---|---|---|---|
| Cell line | ST2897 | ST2898 | ST2899 |
| BMEC | 35 ± 1.8 | 35 ± 0.3 | <<40 |

Tubulin Polymerisation Inhibition Test

The tubulin polymerisation test in the presence of ST2151 was performed as described by Shiff et al. (Biochemistry, 1981, 20: 3247-3252) with a number of modifications. In brief, tubulin rich in microtubule-associated proteins (MAP) was diluted to the concentration of 3 mg/ml in PEM buffer [100 mM PIPES (pH 6.9), 1 mM EGTA and 1 mM MgCl₂] containing 1 mM GTP (GPEM), and maintained in ice. The solution was placed at 37° C. and polymerisation was monitored by measuring absorbance at 340 nm every 25 seconds with a spectrophotometer equipped with an electronic temperature control device (Cobas-Mira Analyzer). After 5 minutes, when the polymerised tubulin had reached a steady state, 5 μM Taxol, 1.35 μM Colcemid, or ST2151 were added and the absorbance measurements were taken for a further 15 min. The IC₅₀ values were determined by non-linear regression analysis using “Prism GraphPad” software. Results were expressed as % inhibition of tubulin polymerization vs not treated control.

The value indicated in Table 2 is the mean of 3 independent determinations.

TABLE 2

| Compound | % inhibition of tubulin polymerization |
|---|---|
| ST2151 | 37.1 |
| ST2179 | 44.0 |

Evaluation of Anticancer Activity

The anticancer activity of ST2495 and ST2496 was assayed in an animal model of human lung carcinoma.

In this model, human NCI-H460 lung cancer cells at a density of 3×10⁶ cells/mouse were injected subcutaneously in the right flank of nude CD1 mice.

Starting from day 4 after tumour cell inoculation, the animals were treated with the study molecules at various doses and according to various treatment schedules (see tables).

All the animals were weighed during the course of the treatment to adjust the drug administration volume and to record the percentage loss of body weight (% BWL).

Tumour growth was assessed by measuring the shorter diameter (width) and the longer diameter (length) of each tumour twice a week with a Vernier caliper, and the anticancer activity was evaluated in terms of percentage inhibition of tumour growth. The tumour volume was calculated using the following formula: tumour volume (TV) in mm³=[length (mm)×width (mm)²]/2. The percentage inhibition (% TVI) was calculated according to the following equation: 100−[(mean tumour volume of the treated group/mean tumour volume of the control group)×100]. A value of P≦0.05 was regarded as statistically significant.

The results of the experimentation with ST2495 and with ST2496 are presented in Tables 3 and 4, respectively.

TABLE 3

| | | | | % TVI | |
|---|---|---|---|---|---|
| | | | | Days after tumor | |
| | | % | | inoculation | |
| Treatment | n | BWL | Mortality | 15 | 22 |
| Vehicle | 8 | 0 | 0/8 | / | / |
| (5% glucose solution) | | | | | |
| ST2495 i.p | 8 | 4 | 0/8 | 32** | 38* |
| 30 mg/kg | | | | | |
| ST2495 i.p | 8 | 0 | 0/8 | 75** | 72** |
| 30 mg/kg twice/day | | | | | |
| Vehicle | 8 | 1 | 0/8 | / | / |
| (5% glucose solution) | | | | | |
| ST2495 p.o | 8 | 0 | 0/8 | 42** | 30* |
| 30 mg/kg twice/day | | | | | |
| ST2495 p.o | 8 | 1 | 0/8 | 79** | 67** |
| 60 mg/kg twice/day | | | | | |
| Vehicle | 8 | 2 | 0/8 | / | / |
| (5% glucose solution) | | | | | |
| ST2495 i.v. | 8 | 1 | 0/8 | 84** | 73** |
| 60 mg/kg | | | | | |
| ST2495 i.v. | 8 | 0 | 0/8 | 81** | 62** |
| 90 mg/kg | | | | | |

ST2495 was given intraperitoneally or orally from day 4 to day 22 according to the schedule qd5x/w once or twice a day, and intravenously from day 4 to day 16 according to the schedule q2dx6.
*P < 0.05, **P < 0.01 (Mann Whithney's test)

TABLE 4

| | | | | % TVI | |
|---|---|---|---|---|---|
| | | | | Days after tumor | |
| | | % | | inoculation | |
| Treatment | N | BWL | Mortality | 14 | 21 |
| Vehicle | 8 | 1 | 0/8 | / | / |
| (saline) | | | | | |
| ST2496 p.o. | 8 | 1 | 0/8 | 48* | 46* |
| 30 mg/kg | | | | | |
| Vehicle | 8 | 1 | 0/8 | / | / |
| (5% glucose | | | | | |
| solution) | | | | | |
| ST2496 i.v. | 8 | 1 | 0/8 | 48** | 53** |
| 60 mg/kg | | | | | |
| ST2496 i.v. | 8 | 1 | 0/8 | 57*** | 56** |
| 90 mg/kg | | | | | |

The compound was administered orally (p.o.) at the doses indicated from day 4 to day 14 after inoculation of the tumour according to the qdx5/w schedule, or intravenously at the doses indicated from day 5 to day 17 according to the q2dx6 schedule.
*P < 0.05; **P < 0.01, ***P < 0.001

As can be seen from the tables, ST2495 proved active with all the administration routes. It is noteworthy that the % of volume inhibition in the i.p. and p.o. treatments increased significantly when the compound was administered twice a day.

ST2496, too, administered orally or intravenously, brought about a significant inhibition of the tumours as compared to controls.

Evaluation of Cardiovascular Parameters

Recent data from a Phase I study have demonstrated that combretastatin A4-P is not avoid of side effects, showing episodes of dose-limiting toxicity, including cases of acute coronary syndrome (Cancer Res., 62:3408-3416, 2002). On the basis of these results and since cardiovascular side effects represent a major issue for an antivascular agent, we decided to study the effect of our selected compounds on cardiovascular parameters.

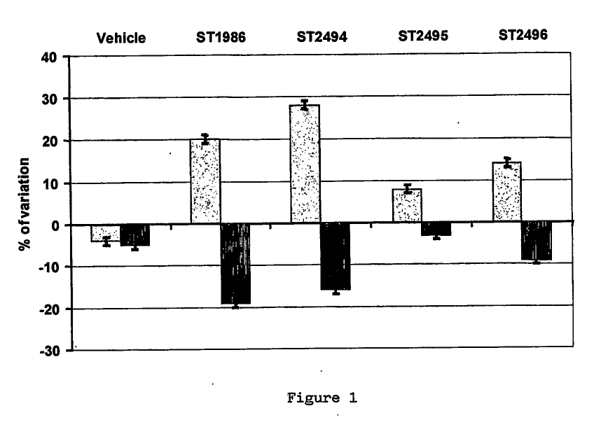

Combretastatin A4, its prodrug ST2494 and our water soluble selected compounds ST2495 and ST2496, diluted at the doses of 20 or 40 mg/kg in saline solution or for combretastatin A4 in 5% DMSO, were injected in the jugular vein of Wistar rats anaesthetised with 55 mg/kg Nembutal. The parameter considered were blood pressure and heart rate. Combretastain A4 and its prodrug ST2494 induced soon after drug administration a significant increase in blood pressure and a progressive decrease of heart rate. In contrast, ST2495 and ST2496 did not show significant effect on the parameter considered (FIG. 1).

In keeping with another object of the present invention, the pharmaceutical compositions contain at least one formula (I) compound as the active ingredient, in an amount such as to produce a significant therapeutic effect without causing cardiovascular side effects. The compositions covered by the present invention are entirely conventional and are obtained using methods which are common practice in the pharmaceutical industry, such as are illustrated, for example, in Reminigton's Pharmaceutical Science Handbook, Mack Pub. N.Y—latest edition.

According to the administration route opted for, the compositions will be in solid or liquid form, suitable for oral, parenteral or intravenous administration. The compositions according to the present invention contain at least one pharmaceutically acceptable vehicle or excipient along with the active ingredient. They may be particularly useful co-adjuvant agents in formulation, e.g. solubilising agents, dispersing agents, suspension agents and emulsifying agents.

1. Formula (I) compounds 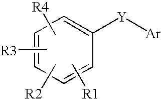
in which
the various R1, R2, R3 and R4, which can be the same or different, are H, OH, OPO3H2 or OCH2OPO3H2 and their disodium salt, OMe, OCH2O, NO2, F, Cl, Br; —R1-R2- can also be together: —CR8=CR9-X—
Y is a group selected from 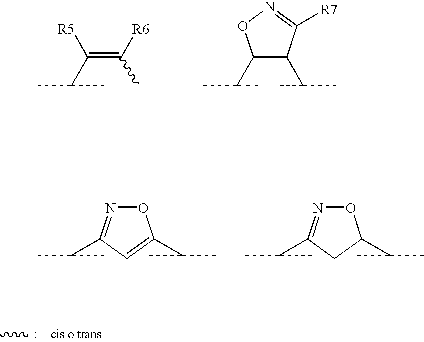
R5 and R6, which can be the same or different, are H or halogen;
R7 is H, OMe, SO2Ph;
Ar is a group selected from: 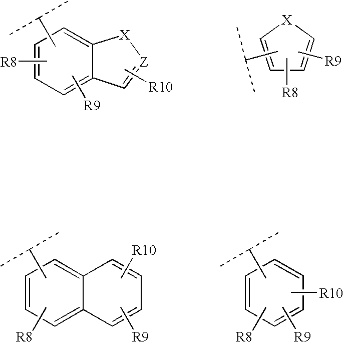
R8, R9 and R10, which can be the same or different, are H, OH, OPO3H2 or OCH2OPO3H2 and their disodium salt, OR11, OCH2O, NH2, NHR11, NO2, alkyl (C1-C4), C6H5, C5H4N or halogen;
R11 is C1-C4 alkyl or acyl, aminoacids residue;
X is O, S, N, NR12;
R12 is H, CH3, CH2Ph;
Z is CH, N;
with the proviso that the formula (I) compound is not combretastatin A-1, combretastatin A-2, combretastatin A-4, and their disodium phosphates derivatives and with the exclusion of the following compounds:
2-phenyl-6-trans-styryl-benzo[b]furan;
2,3-diphenyl-6-trans-styryl-benzo[b]furan;
2-phenyl-6-(4-methoxy)-trans-styryl-benzo[b]furan;
2-phenyl-6-(3,4-dimethoxy)-trans-styryl-benzo[b]furan;
2-phenyl-6-(3,4,5-trimethoxy)-trans-styryl-benzo[b]furan;
2-phenyl-6-(3,4-methylenedioxy)-trans-styryl-benzo[b]furan;
2,3-diphenyl-6-(4-methoxy)-trans-styryl-benzo[b]furan;
2-phenyl-5-trans-styryl-benzo[b]thiophene;
2-phenyl-5-(4-methoxy)-trans-styryl-benzo[b]thiophene;
2-phenyl-5-(3,4-methylenedioxy)-trans-styryl-benzo[b]thiophene;
2-phenyl-6-trans-styryl-benzo[b]thiophene;
2-phenyl-6-(4-methoxy)-trans-styryl-benzo[b]thiophene;
2-phenyl-6-(4-chloro)-trans-styryl-benzo[b]thiophene;
Piceatannol;
1-(3-furanyl)-2-(3,4,5-trimethoxyphenyl)ethene;
1-(3-thiophenyl)-2-(3,4,5-trimethoxyphenyl)ethene;
1-(2-furanyl)-2-(3,4,5-trimethoxyphenyl)ethene;
and with the proviso that
when R1 is hydrogen and R2-R4 are 3,4,5-trimethoxy, Y is a double bond, R5 and R6 are H, Ar is phenyl, R8 and R9 are hydrogen, R10 is not methoxy;
when R1 is hydrogen and R2-R4 are 3,4,5-trimethoxy, Y is a double bond, R5 and R6 are H, Ar is phenyl, R8 is hydrogen, R9 is 2-chloro, R10 is not 4-methoxy;
when R1 is hydrogen and R2-R4 are trimethoxy, Y is a double bond, R5 and R6 are H, Ar is phenyl, at least one of R8-R10 is not hydrogen;
when R1 is hydrogen and R2-R4 are 3,4,5-trimethoxy, Y is a double bond, R5 and R6 are H, Ar is phenyl, R8 and R9 are hydrogen, R10 is none of 4-chloro, 4-bromo, 4-nitro, 4-hydroxy, 4-acetyl, 4-ethoxy, 4-C1-C4 alkyl;
when R1 is hydrogen and R2-R4 are 3,4,5-trimethoxy, Y is a double bond, R5 and R6 are H, Ar is phenyl, R8 is hydrogen, R9 is 4-nitro or 4-amino, R10 is none of 3-chloro, 3-methoxy, 3-methyl;
when R1 is hydrogen and R2-R4 are 3,4,5-trimethoxy, Y is a cis double bond, R5 and R6 are H, Ar is phenyl, R8 is hydrogen, R9 is 3-nitro or 3-amino, R10 is none of 3-chloro, 3-methoxy, 3-methyl;
when R1 is hydrogen and R2-R4 are 2,3,4-trimethoxy, Y is a double bond, R5 and R6 are H, Ar is phenyl, R8 and R9 are hydrogen, R10 is not 4-methoxy;
when R1 is hydrogen and R2-R4 are 3,4,5-trimethoxy, Y is a double bond, R5 and R6 are H, Ar is phenyl, at least one of R8 is hydrogen, R9 is 3-methoxy, R10 is not 5-methoxy;
when R1 is hydrogen and R2-R4 are 3,4,5-trimethoxy, Y is a double bond, R5 and R6 are H, Ar is phenyl, R8-R10 are not methoxy;
when R1 and R2 are hydrogen and R3-R4 are 3,4-dimethoxy, Y is a double bond, R5 and R6 are H, Ar is phenyl, R8 and R9 are hydrogen, R10 is not 4-methoxy;
when R1 and R2 are hydrogen and R3-R4 are 3,4-dimethoxy, Y is a double bond, R5 and R6 are H, Ar is phenyl, R8 is hydrogen, R9-R10 are not 3,5-dimethoxy;
when R1 and R2 are hydrogen and R3-R4 are 3,4-dimethoxy, Y is a double bond, R5 and R6 are H, Ar is phenyl, at least one of R8-R10 is not hydrogen;
when R1 and R2 are hydrogen and R3-R4 are 3,5-methoxy, Y is a double bond, R5 and R6 are H, Ar is phenyl, R8 and R9 are hydrogen, R10 is not 4-methoxy;
when R1 and R2 are hydrogen and R3-R4 are 3,5-methoxy, Y is a double bond, R5 and R6 are H, Ar is phenyl, R8 and R9 are hydrogen, R10 is not 4-acetyl;
when R1 is hydrogen and R2-R4 are 3,4,5-trimethoxy, Y is a double bond, R5 and R6 are H, Ar is not pyridyl;
when R1 is hydrogen and R2-R4 are 3,4,5-trimethoxy, Y is cis double bond, R5 and R6 are H, Ar is phenyl, R8 is hydrogen, R9 is 3-amino, R10 is 4-NHR11, R11 is not the residue of serine;
when R1 is hydrogen and R2-R4 are 3,4,5-trimethoxy, Y is cis double bond, R5 and R6 are H, Ar is phenyl, R8 is hydrogen, R9 is 3-amino, R10 is not 4-methoxy;
when R1 is hydrogen and R2-R4 are 3,4,5-trimethoxy, Y is cis double bond, R5 and R6 are H, Ar is phenyl, R8 is hydrogen, R9 is 3-amino, R10 is not a 4-alkyloxy group having from 1 to 3 carbon atoms, or a 4-alkyl group having from 1 to 4 carbon atoms, or a halogen atom
when R1 is hydrogen and R2-R3 are 3,4-methylenedioxy, R4 is 5-methoxy, Y is cis double bond, R5 and R6 are H, Ar is phenyl, R8 is hydrogen, R9 is 3-amino, R10 is not 4-methoxy;
when R1 is hydrogen and R2-R4 are 2,3,4-trimethoxy, Y is cis double bond, R5 and R6 are H, Ar is phenyl, R8 is hydrogen, R9 is 3-amino, R10 is not 4-methoxy;
when R1 is hydrogen and R2-R4 are 3,4,5-trimethoxy, Y is cis double bond, R5 and R6 are H, Ar is phenyl, R8 is hydrogen, R9 is NHR11, R11 is the residue of serine, R10 is not 4-methoxy;
when R1 is hydrogen and R2-R3 are 3,4-methylenedioxy, R4 is 4-methoxy, Y is cis double bond, R5 and R6 are H, Ar is phenyl, R8 is hydrogen, R9 is NHR11, R11 is the residue of the aminoacid cysteine, glycine, phenylalanine, serine, triptophan, tyrosine, valine, R10 is not 4-methoxy;
when R1 is hydrogen and R2-R3 are 3,4-methylenedioxy, R4 is 4-methoxy, Y is a double bond, R5 and R6 are H, Ar is phenyl, R8 is hydrogen, R9 is NO2 or NH2, R10 is not 4-methoxy;
when R1 is hydrogen and R2-R4 are 3,4,5-trimethoxy, Y is cis double bond, R5 and R6 are H, Ar is phenyl, at least one of R8-R10 is not hydrogen;
when R1 is hydrogen and R2-R4 are 3,4,5-trimethoxy, Y is a double bond, R5 and R6 are H, Ar is phenyl, R8 is hydrogen, R9 is 4-methoxy, R10 is not 3-fluoro;
when R1 is hydrogen and R2-R4 are 3,4,5-trimethoxy, Y is a double bond, R5 and R6 are H, Ar is phenyl, R8 is hydrogen, R9 is 4-methyl, R10 is not 3-fluoro or 3-hydroxy;
when R1 is hydrogen and R2-R4 are 3,4,5-trimethoxy, Y is cis double bond, R5 and R6 are H, Ar is phenyl, R8 is hydrogen, R9 is 4-methoxy, R10 is not 3-methoxy;
when R1 is hydrogen and R2-R4 are 3,4,5-trimethoxy, Y is cis double bond, R5 and R6 are H, Ar is phenyl, R8 is 3-fluoro, R9 is 4-methoxy, R10 is not 2- or 5-fluoro;
when R1 is hydrogen and R2-R4 are 3,4,5-trimethoxy, Y is cis double bond, R5 and R6 are H, Ar is phenyl, R8 is hydrogen, R9 is 4-methoxy, R10 is not 3-hydroxy or 3-amino;
when R1 is hydrogen and R2-R4 are 3,4,5-trimethoxy, Y is cis double bond, R5 and R6 are H, Ar is phenyl, R8 is hydrogen, R9 is 4-methoxy, R10 is not 3-fluoro or 3-bromo;
when R1 is hydrogen and R2-R4 are 3,4,5-trimethoxy, Y is cis double bond, R5 and R6 are H, Ar is phenyl, R8 and R9 are hydrogen, R10 is not 4-hydroxy;
when R1 is hydrogen and R2-R4 are 3,4,5-trimethoxy, Y is cis double bond, R5 and R6 are H, Ar is phenyl, R8 is hydrogen, R9 is 3-methyl, R10 is not 4-methyl;
when R1 is hydrogen and R2-R4 are 3,4,5-trimethoxy, Y is cis double bond, R5 and R6 are H, Ar is phenyl, R8 is hydrogen, R9 is 4-methoxy, R10 is not 3-hydroxy;
when R1-R2 are hydrogen and R3-R4 are 3,5-dihydroxy, Y is trans double bond, R5 and R6 are H, Ar is phenyl, R8 is hydrogen, R9 is 3-hydroxy, R10 is not 5-hydroxy;
when R1-R3 are hydrogen, Y is a double bond, R5 and R6 are H, Ar is phenyl, R8 is hydrogen, R9 and R10 are 3,4-dimethyl, and R4 is not 4-methoxy;
when R1-R2 are hydrogen, Y is a double bond, R5 and R6 are H, Ar is phenyl, R8 is hydrogen, R9 and R10 are 3,4-dimethyl, R4 is 4-methoxy, R3 is not 3-fluoro or 3-bromo or 3-nitro or 3-hydroxy;
when R1-R2 are hydrogen, Y is a double bond, R5 and R6 are H, Ar is phenyl, R8-R10 are 3,4,5-triethoxy, R4 is 4-methoxy, R3 is not 3-fluoro or 3-chloro or 3-bromo or 3-hydroxy;
when R1-R2 are hydrogen, R4 is 4-methoxy, Y is a double bond, R5 and R6 are H, Ar is phenyl, R8-R9 are 4,5-dimethoxy, R10 is 3-hydroxy, R3 is not 3-fluoro or 3-hydroxy;
when R1-R2 are hydrogen, R4 is 4-methoxy, Y is a double bond, R5 and R6 are H, Ar is phenyl, R8-R9 are 4,5-dimethoxy, R10 is 3-methoxy, R3 is not 3-fluoro;
when R1 is hydrogen, R2-R4 are 3,4,5-trimethoxy, Y is a double bond, R5 and R6 are H, Ar is 2-naphthyl, at least one of R8-R10 is not hydrogen;
when R1 and R2 are hydrogen, R3 is 3-hydroxy, R4 is 4-methoxy, Y is a double bond, R5 and R6 are H, Ar is 2-naphthyl, at least one of R8-R10 is not hydrogen;
when R1 is hydrogen, R2-R4 are 3,4,5-trimethoxy, Y is 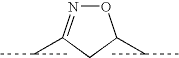
Ar is indolyl, wherein at least one of R8-R10 is different from hydrogen;
their enantiomers, diastereoisomers, the respective mixtures and their pharmaceutically acceptable salts.

2. Compound according to claim 1, selected from the group consisting of:
2-methoxy-5-[3-methoxy-5-(3,4,5-trimethoxy-phenyl)-4,5-dihydro-4-isoxazolyl]-phenol;
2-methoxy-5-[3-methoxy-4-(3,4,5-trimethoxy-phenyl)-4,5-dihydro-5-isoxazolyl]-phenol;
5-[3-benzenesulphonyl-4-(3,4,5-trimethoxy-phenyl)-4,5-dihydro-4-isoxazolyl]-2-methoxy-phenol;
5-[3-benzenesulphonyl-5-(3,4,5-trimethoxy-phenyl)-4,5-dihdro-5-isoxazolyl]-2-methoxy-phenol;
2-methoxy-5-[3-(3,4,5-trimethoxy-phenyl)-4,5-dihydro-5-isoxazolyl]-phenol;
2-methoxy-5-[5-(3,4,5-trimethoxy-phenyl)-4,5-dihydro-3-isoxazolyl]-phenol;
2-methoxy-5-[5-(3,4,5-trimethoxy-phenyl)-3-isoxazole]-phenol;
cis-6-[2-(3,4,5-trimethoxy-phenyl)-vinyl]-benzo[b]thiophen-4-ol;
trans-6-[2-(3,4,5-trimethoxy-phenyl)-vinyl]-benzo[b]thio-phen-4-ol;
cis-4-methoxy-6-[2-(3,4,5-trimethoxy-phenyl)-vinyl]-benzo[b]thiophene;
trans-4-methoxy-6-[2-(3,4,5-trimethoxy-phenyl)-vinyl]-benzo[b]thio-phene;
cis-6-[2-(3,4,5-trimethoxy-phenyl)-vinyl]-benzofuran-4-ol;
trans-6-[2-(3,4,5-trimethoxy-phenyl)-vinyl]-benzofuran-4-ol;
cis-4-methoxy-6-[2-(3,4,5-trimethoxy-phenyl)-vinyl]-benzofuran;
trans-4-methoxy-6-[2-(3,4,5-trimethoxy-phenyl)-vinyl]-benzofuran;
cis-5-[2-(3,4,5-trimethoxy-phenyl)-vinyl]-benzo[b]thiophen-7-ol;
trans-5-[2-(3,4,5-trimethoxy-phenyl)-vinyl]-benzo[b]thiophen-7-ol;
cis-5-[2-(3,4,5-trimethoxy-phenyl)-vinyl]-benzofuran-7-ol;
trans-5-[2-(3,4,5-trimethoxy-phenyl)-vinyl]-benzofuran-7-ol;
cis-1-methoxy-3-[2-(3,4,5-trimethoxy-phenyl)-vinyl]-naphthalene;
methoxy-3-[2-(3,4,5-trimethoxy-phenyl)-vinyl]-naphthalene;
cis-7-methoxy-1-methyl-5-[2-(3,4,5-trimethoxy-phenyl)-vinyl]-1H-in-dazole;
trans-7-methoxy-1-methyl-5-[2-(3,4,5-trimethoxy-phenyl)-vinyl]-1H-indazole;
2-nitro-5-[2-(3,4,5-trimethoxy-phenyl)-vinyl]-thiophene;
2-nitro-5-[2-(3,4,5-trimethoxy-phenyl)-vinyl]-furan;
cis-3-[2-(3,4,5-trimethoxy-phenyl)-vinyl]-naphthalen-1-ol;
trans-3-[2-(3,4,5-trimethoxy-phenyl)-vinyl]-naphthalen-1-ol;
disodium 6[(Z)-2-(3,4,5-trimethoxy-phenyl)ethenyl]-1-benzo-thiophen-4-ol 4-O-phosphate;
disodium 6[(Z)-2-(3,4,5-trimethoxyphenyl)ethenyl]-1-benzo-furan-4-ol 4-O-phosphate;
6-[(Z)-2-(7-methoxy-1,3-benzodioxol-5-yl)vinyl]-1-benzothiophene-4-ol;
6-[(E)-2-(7-methoxy-1,3-benzodioxol-5-yl)vinyl]-1-benzothiophene-4-ol;
6[(Z)-2-(3-methoxy-4,5-metilendioxy-phenyl-1-yl)-vinyl]-1-benzofuran-4-ol;
6[(E)-2-(3-methoxy-4,5-metilendioxy-phenyl-1-yl)-vinyl]-1-benzofuran-4-ol;
disodium 6[(Z)-2-(3,4,5-trimethoxy-phenyl)ethenyl]-1-benzo-thiophen-4-ol 4-O-methyloxyphosphate;
disodium 6[(Z)-2-(3,4,5-trimethoxyphenyl)ethenyl]-1-benzo-furan-4-ol 4-O-methyloxyphosphate;
6-[(Z)-2-(7-methoxy-1,3-benzodioxol-5-yl)vinyl]-1-benzothiophene-4-ol;
6-[(E)-2-(7-methoxy-1,3-benzodioxol-5-yl)vinyl]-1-benzothiophene-4-ol.
6[(2)-2-(3-methoxy-4,5-metilendioxy-phenyl-1-yl)-vinyl]-1-benzofuran-4-ol;
6[(E)-2-(3-methoxy-4,5-methylenedioxy-phenyl-1-yl)-vinyl]-1-benzofu-ran-4-ol;
disodium 6[(Z)-2-(3,4,5-trimethoxy-phenyl)ethenyl]-1-benzo-thiophen-4-ol 4-O-methyloxyphosphate;
disodium 6[(Z)-2-(3,4,5-trimethoxyphenyl)ethenyl]-1-benzo-furan-4-ol 4-O-methyloxyphosphate;
6-[(Z)-2-(7-methoxy-1,3-benzodioxol-5-yl)vinyl]-1-benzothiophene-4-ol;
cis-2-Methoxy-5-[2-(4-methoxy-benzofuran-6-yl)-vinyl]-phenol;
cis-2-Methoxy-5-[2-(7-methoxy-benzofuran-5-yl)-vinyl]-phenol;
cis-2-Methoxy-5-[2-(4-methoxy-benzo[b]thiophen-6-yl)-vinyl]-phenol;
cis-6-[2-(3,5-dimethoxy-phenyl)-vinyl]-benzo[b]thiophen-4-ol;
cis-5-[2-(3,5-dimethoxy-phenyl)-vinyl]-benzofuran-7-ol;
cis-6-[2-(3,5-dimethoxy-phenyl)-vinyl]-benzofuran-4-ol;
their enantiomers, diastereoisomers, the respective mixtures and their pharmaceutically acceptable salts.

3. Use of formula (I) compounds 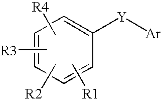
in which
the various R1, R2, R3 and R4, which can be the same or different, are H, OH, OPO3H2 or OCH2OPO3H2 and their disodium salt, OMe, OCH2O, NO2, F, Cl, Br; —R1-R2- can also be together: —CR8=CR9-X—
Y is a group selected from 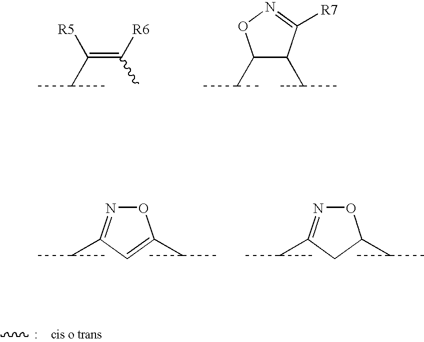
R5 and R6, which can be the same or different, are H or halogen;
R7 is H, OMe, SO2Ph;
Ar is a group selected from: 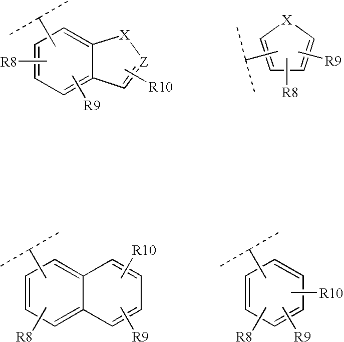
R8, R9 and R10, which can be the same or different, are H, OH, OPO3H2 or OCH2OPO3H2 and their disodium salt, OR11, OCH2O, NH2, NHR11, NO2, alkyl (C1-C4), C6H5, C5H4N or halogen;
R11 is C1-C4 alkyl or acyl, aminoacids residue;
X is O, S, N, NR12;
R12 is H, CH3, CH2Ph;
Z is CH, N;
with the proviso that the formula (I) compound is not combretastatin A-1, combretastatin A-2, combretastatin A-4, and their disodium phosphates derivatives and with the exclusion of the following compounds:
Piceatannol;
1-(3-furanyl)-2-(3,4,5-trimethoxyphenyl)ethene;
1-(3-thiophenyl)-2-(3,4,5-trimethoxyphenyl)ethene;
1-(2-furanyl)-2-(3,4,5-trimethoxyphenyl)ethene;
and with the proviso that
when R1 is hydrogen and R2-R4 are 3,4,5-trimethoxy, Y is a double bond, R5 and R6 are H, Ar is phenyl, R8 and R9 are hydrogen, R10 is not methoxy;
when R1 is hydrogen and R2-R4 are 3,4,5-trimethoxy, Y is a double bond, R5 and R6 are H, Ar is phenyl, R8 is hydrogen, R9 is 2-chloro, R10 is not 4-methoxy;
when R1 is hydrogen and R2-R4 are trimethoxy, Y is a double bond, R5 and R6 are H, Ar is phenyl, at least one of R8-R10 is not hydrogen;
when R1 is hydrogen and R2-R4 are 3,4,5-trimethoxy, Y is a double bond, R5 and R6 are H, Ar is phenyl, R8 and R9 are hydrogen, R10 is none of 4-chloro, 4-bromo, 4-nitro, 4-hydroxy, 4-acetyl, 4-ethoxy, 4-C1-C4 alkyl;
when R1 is hydrogen and R2-R4 are 3,4,5-trimethoxy, Y is a double bond, R5 and R6 are H, Ar is phenyl, R8 is hydrogen, R9 is 4-nitro or 4-amino, R10 is none of 3-chloro, 3-methoxy, 3-methyl;
when R1 is hydrogen and R2-R4 are 3,4,5-trimethoxy, Y is a cis double bond, R5 and R6 are H, Ar is phenyl, R8 is hydrogen, R9 is 3-nitro or 3-amino, R10 is none of 3-chloro, 3-methoxy, 3-methyl;
when R1 is hydrogen and R2-R4 are 2,3,4-trimethoxy, Y is a double bond, R5 and R6 are H, Ar is phenyl, R8 and R9 are hydrogen, R10 is not 4-methoxy;
when R1 is hydrogen and R2-R4 are 3,4,5-trimethoxy, Y is a double bond, R5 and R6 are H, Ar is phenyl, at least one of R8 is hydrogen, R9 is 3-methoxy, R10 is not 5-methoxy;
when R1 is hydrogen and R2-R4 are 3,4,5-trimethoxy, Y is a double bond, R5 and R6 are H, Ar is phenyl, R8-R10 are not methoxy;
when R1 and R2 are hydrogen and R3-R4 are 3,4-dimethoxy, Y is a double bond, R5 and R6 are H, Ar is phenyl, R8 and R9 are hydrogen, R10 is not 4-methoxy;
when R1 and R2 are hydrogen and R3-R4 are 3,4-dimethoxy, Y is a double bond, R5 and R6 are H, Ar is phenyl, R8 is hydrogen, R9-R10 are not 3,5-dimethoxy;
when R1 and R2 are hydrogen and R3-R4 are 3,4-dimethoxy, Y is a double bond, R5 and R6 are H, Ar is phenyl, at least one of R8-R10 is not hydrogen;
when R1 and R2 are hydrogen and R3-R4 are 3,5-methoxy, Y is a double bond, R5 and R6 are H, Ar is phenyl, R8 and R9 are hydrogen, R10 is not 4-methoxy;
when R1 and R2 are hydrogen and R3-R4 are 3,5-methoxy, Y is a double bond, R5 and R6 are H, Ar is phenyl, R8 and R9 are hydrogen, R10 is not 4-acetyl;
when R1 is hydrogen and R2-R4 are 3,4,5-trimethoxy, Y is a double bond, R5 and R6 are H, Ar is not pyridyl;
when R1 is hydrogen and R2-R4 are 3,4,5-trimethoxy, Y is cis double bond, R5 and R6 are H, Ar is phenyl, R8 is hydrogen, R9 is 3-amino, R10 is 4-NHR11, R11 is not the residue of serine;
when R1 is hydrogen and R2-R4 are 3,4,5-trimethoxy, Y is cis double bond, R5 and R6 are H, Ar is phenyl, R8 is hydrogen, R9 is 3-amino, R10 is not 4-methoxy;
when R1 is hydrogen and R2-R4 are 3,4,5-trimethoxy, Y is cis double bond, R5 and R6 are H, Ar is phenyl, R8 is hydrogen, R9 is 3-amino, R10 is not a 4-alkyloxy group having from 1 to 3 carbon atoms, or a 4-alkyl group having from 1 to 4 carbon atoms, or a halogen atom
when R1 is hydrogen and R2-R3 are 3,4-methylenedioxy, R4 is 5-methoxy, Y is cis double bond, R5 and R6 are H, Ar is phenyl, R8 is hydrogen, R9 is 3-amino, R10 is not 4-methoxy;
when R1 is hydrogen and R2-R4 are 2,3,4-trimethoxy, Y is cis double bond, R5 and R6 are H, Ar is phenyl, R8 is hydrogen, R9 is 3-amino, R10 is not 4-methoxy;
when R1 is hydrogen and R2-R4 are 3,4,5-trimethoxy, Y is cis double bond, R5 and R6 are H, Ar is phenyl, R8 is hydrogen, R9 is NHR11, R11 is the residue of serine, R10 is not 4-methoxy;
when R1 is hydrogen and R2-R3 are 3,4-methylenedioxy, R4 is 4-methoxy, Y is cis double bond, R5 and R6 are H, Ar is phenyl, R8 is hydrogen, R9 is NHR11, R11 is the residue of the aminoacid cysteine, glycine, phenylalanine, serine, triptophan, tyrosine, valine, R10 is not 4-methoxy;
when R1 is hydrogen and R2-R3 are 3,4-methylenedioxy, R4 is 4-methoxy, Y is a double bond, R5 and R6 are H, Ar is phenyl, R8 is hydrogen, R9 is NO2 or NH2, R10 is not 4-methoxy;
when R1 is hydrogen and R2-R4 are 3,4,5-trimethoxy, Y is cis double bond, R5 and R6 are H, Ar is phenyl, at least one of R8-R10 is not hydrogen;
when R1 is hydrogen and R2-R4 are 3,4,5-trimethoxy, Y is a double bond, R5 and R6 are H, Ar is phenyl, R8 is hydrogen, R9 is 4-methoxy, R10 is not 3-fluoro;
when R1 is hydrogen and R2-R4 are 3,4,5-trimethoxy, Y is a double bond, R5 and R6 are H, Ar is phenyl, R8 is hydrogen, R9 is 4-methyl, R10 is not 3-fluoro or 3-hydroxy;
when R1 is hydrogen and R2-R4 are 3,4,5-trimethoxy, Y is cis double bond, R5 and R6 are H, Ar is phenyl, R8 is hydrogen, R9 is 4-methoxy, R10 is not 3-methoxy;
when R1 is hydrogen and R2-R4 are 3,4,5-trimethoxy, Y is cis double bond, R5 and R6 are H, Ar is phenyl, R8 is 3-fluoro, R9 is 4-methoxy, R10 is not 2- or 5-fluoro;
when R1 is hydrogen and R2-R4 are 3,4,5-trimethoxy, Y is cis double bond, R5 and R6 are H, Ar is phenyl, R8 is hydrogen, R9 is 4-methoxy, R10 is not 3-hydroxy or 3-amino;
when R1 is hydrogen and R2-R4 are 3,4,5-trimethoxy, Y is cis double bond, R5 and R6 are H, Ar is phenyl, R8 is hydrogen, R9 is 4-methoxy, R10 is not 3-fluoro or 3-bromo;
when R1 is hydrogen and R2-R4 are 3,4,5-trimethoxy, Y is cis double bond, R5 and R6 are H, Ar is phenyl, R8 and R9 are hydrogen, R10 is not 4-hydroxy;
when R1 is hydrogen and R2-R4 are 3,4,5-trimethoxy, Y is cis double bond, R5 and R6 are H, Ar is phenyl, R8 is hydrogen, R9 is 3-methyl, R10 is not 4-methyl;
when R1 is hydrogen and R2-R4 are 3,4,5-trimethoxy, Y is cis double bond, R5 and R6 are H, Ar is phenyl, R8 is hydrogen, R9 is 4-methoxy, R10 is not 3-hydroxy;
when R1-R2 are hydrogen and R3-R4 are 3,5-dihydroxy, Y is trans double bond, R5 and R6 are H, Ar is phenyl, R8 is hydrogen, R9 is 3-hydroxy, R10 is not 5-hydroxy;
when R1-R3 are hydrogen, Y is a double bond, R5 and R6 are H, Ar is phenyl, R8 is hydrogen, R9 and R10 are 3,4-dimethyl, and R4 is not 4-methoxy;
when R1-R2 are hydrogen, Y is a double bond, R5 and R6 are H, Ar is phenyl, R8 is hydrogen, R9 and R10 are 3,4-dimethyl, R4 is 4-methoxy, R3 is not 3-fluoro or 3-bromo or 3-nitro or 3-hydroxy;
when R1-R2 are hydrogen, Y is a double bond, R5 and R6 are H, Ar is phenyl, R8-R10 are 3,4,5-triethoxy, R4 is 4-methoxy, R3 is not 3-fluoro or 3-chloro or 3-bromo or 3-hydroxy;
when R1-R2 are hydrogen, R4 is 4-methoxy, Y is a double bond, R5 and R6 are H, Ar is phenyl, R8-R9 are 4,5-dimethoxy, R10 is 3-hydroxy, R3 is not 3-fluoro or 3-hydroxy;
when R1-R2 are hydrogen, R4 is 4-methoxy, Y is a double bond, R5 and R6 are H, Ar is phenyl, R8-R9 are 4,5-dimethoxy, R10 is 3-methoxy, R3 is not 3-fluoro;
when R1 is hydrogen, R2-R4 are 3,4,5-trimethoxy, Y is a double bond, R5 and R6 are H, Ar is 2-naphthyl, at least one of R8-R10 is not hydrogen;
when R1 and R2 are hydrogen, R3 is 3-hydroxy, R4 is 4-methoxy, Y is a double bond, R5 and R6 are H, Ar is 2-naphthyl, at least one of R8-R10 is not hydrogen;
when R1 is hydrogen, R2-R4 are 3,4,5-trimethoxy,
Y is 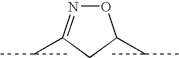
Ar is indolyl, wherein at least one of R8-R10 is different from hydrogen;
their enantiomers, diastereoisomers, the respective mixtures and their pharmaceutically acceptable salts as medicaments.

4. Use according to claim 3 for the preparation of a medicament for the treatment of oncological-type diseases.

5. Use according to claim 3 for the preparation of a medicament for the treatment of cancers that respond to cytotoxic activity.

6. Use according to claim 5, in which said cancer is selected from the group consisting of sarcoma, carcinoma, carcinoid, bone cancer, neuroendocrine cancer, lymphoid leukaemia, myeloid leukaemia, monocytic leukaemia, megakaryocytic leukaemia, or Hodgkin's disease.

7. Use of compounds according to claim 1 for the preparation of a medicament for the treatment of diseases related to abnormal angiogenesis.

8. Use according to claim 7, in which said disease is selected from the group consisting of arthritic disease, tumours responding to antiangiogenic activity, metastatic spread, diabetic retinopathy, psoriasis, chronic inflammation, and atherosclerosis.

9. Use according to claim 4, in which, in the treatment of tumours, said medicament is combined with at least one other antiblastic drug.

10. Use according to claim 9, in which said antiblastic drug is selected from the group consisting of alkylating agents; topoisomerase inhibitors; antitubulin agents; intercalating agents; antimetabolites; naturally occurring products such as Vinca alkaloids, epipodophyllotoxins, antibiotics, enzymes, taxanes and anticancer vaccines.

11. Pharmaceutical composition containing as the active ingredient a compound according to claim 1 in a mixture with a pharmaceutically acceptable excipient or diluent.

12. Use of the compound with the formula 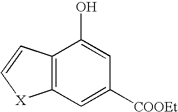
in which
X is oxygen or sulphur, as an intermediate product for the preparation of compounds according to claim 1.

13. Compound with the formula: 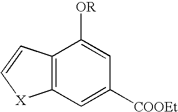
in which
X is oxygen or sulphur, R is methyl, or terbutyl-dimethylsilyl.

14. Compound with the formula 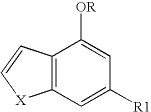
in which
X is oxygen or sulphur, R is methyl, or terbutyl-dimethylsilyl.
R1 is formyl.

15. Use of the compound with the formula 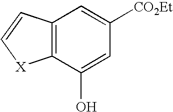
in which
X is oxygen or sulphur, as an intermediate product for the preparation of compounds according to claim 1.

16. Compound with the formula 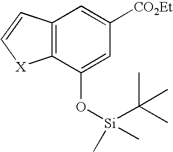
in which
X is oxygen or sulphur.

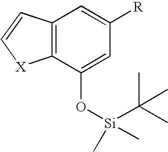

17. Compound with the formula
in which
X is oxygen or sulphur.

18. (canceled)
